(19) United States
(12) Patent Application Publication
(10) Pub. No.: US 2014/0081153 A1
(43) Pub. Date: Mar. 20, 2014

(54) PULSE DATA DETECTING APPARATUS, PULSE DATA DETECTING METHOD, AND STORAGE MEDIUM HAVING PULSE DATA DETECTION PROGRAM RECORDED THEREON

(71) Applicant: Casio Computer Co., Ltd., Tokyo (JP)

(72) Inventor: Toshiya KUNO, Tokyo (JP)

(73) Assignee: CASIO COMPUTER CO., LTD., Tokyo (JP)

(21) Appl. No.: 14/021,905

(22) Filed: Sep. 9, 2013

(30) Foreign Application Priority Data
Sep. 18, 2012 (JP) 2012-204103

(51) Int. Cl. A61B 5/024

(52) U.S. Cl. CPC A61B 5/024
USPC 600/479

(57) ABSTRACT

A pulse data detecting apparatus, pulse data detecting method and pulse data detection program are provided capable of suppressing an influence of the condition of the body surface to be measured and obtaining an appropriate measurement result under a wide range of conditions. In the present invention, light-emitting elements irradiate a skin surface with light. A light-emission driving section controls lighting-up and the light emission amount of the plurality of light-emitting elements under the control of a CPU. Light-receiving elements each receive reflected light when the skin surface is irradiated by the light-emitting elements, and output a signal. The CPU determines an appropriate combination of light-emitting elements and a light-receiving element(s) based on the output signal from each of the light-receiving elements. A pulse rate calculating section calculates a pulse rate based on the signal outputted from any of the light-receiving elements in the appropriate combination.

### CROSS-REFERENCE TO RELATED APPLICATIONS

This application is based upon and claims the benefit of priority from the prior Japanese Patent Application No. 2012-204103, filed Sep. 18, 2012, and No. 2013-141224, filed Jul. 5, 2013, the entire contents of which are incorporated herein by reference.

### BACKGROUND OF THE INVENTION

1. Field of the Invention

The present invention relates to a pulse data detecting apparatus mounted on a human body to measure pulse data, a pulse data detecting method, and the like.

2. Description of the Related Art

Conventionally, various types have been available for apparatuses of measuring pulse of a human body. By way of example, a method of obtaining an electrical signal flowing at both ends of the trunk across the heart (an application of an electrocardiogram) and a method of measuring the sound of heartbeat together with measuring a blood pressure are known. Also, based on the fact that the light absorption amount changes with change in concentration (density) of hemoglobin flowing through capillary vessels distributed over the body surface, a (so-called optical) method of using the principle that the light amount of reflected light changes with heartbeat is known as another example of the method for measuring pulses. In this method, the human skin is irradiated with light such as visible light (green or red) or near-infrared light and a change in body-surface reflected light or a change in absorption light amount of hemoglobin by body transmission light is measured.

Among these various types of measuring devices, a scheme called an optical type has been disclosed in, for example, Japanese Patent Application Laid-Open (Kokai) Publication No. 2008-212258. The above-described patent document discloses a laser blood flow meter (pulsimeter) which arranges a plurality of light-emitting elements around one light-receiving element and, determines an optimum one of the plurality of light-emitting elements based on a detection signal obtained at the light-receiving element by individually driving each of the light-emitting elements, whereby positioning on the living body can be easily made and detection accuracy is improved.

Meanwhile, the measurement device disclosed in the above-described patent document, etc., is influenced by the condition of the body surface to be measured, for example, uncertainties such as unevenness in distribution of lentigines (moles), body hair, body color, capillary vessels on the skin surface. As a result, extremely large unevenness may occur in the measurement result. Accordingly, there is a problem such that measurement cannot be performed except in an extremely limited range such as an earlobe or a fingertip.

### SUMMARY OF THE INVENTION

In light of the above-described problems, an object of the present invention is to provide a pulse data detecting apparatus, pulse data detecting method and a pulse data detection program capable of suppressing an influence of the condition of the body surface to be measured and obtaining an appropriate measurement result under a wide range of conditions.

A pulse data detecting apparatus according to the present invention comprising: a plurality of light-emitting elements which irradiate a body to be measured with light; a light emission control section which performs control of causing the plurality of light-emitting elements to emit light in a plurality of light emission patterns; a light-receiving element which receives reflected light when the body to be measured is irradiated by the plurality of light-emitting elements in the plurality of light emission patterns and outputs a signal for each of the light emission patterns; a combination determining section which determines, as an appropriate combination, a combination of any of the plurality of light emission patterns and the light-receiving element satisfying an adequate condition, based on the signal outputted from the light-receiving element; and a pulse data output section which outputs pulse data based on the signal outputted from the light-receiving element, by the combination of the light emission patterns and the light-receiving element determined by the combination determining section as the appropriate combination.

A pulse data detecting apparatus according to the present invention comprising: a plurality of light-emitting elements which irradiate a body to be measured with light; a light emission control section which performs control of light emission amounts of the plurality of light-emitting elements; a light-receiving element which receives reflected light when the body to be measured is irradiated by the plurality of light-emitting elements with the light emission amounts controlled by the light emission control section and outputs a signal; and a pulse data output section which outputs pulse data based on the signal outputted from the light-receiving element.

The above and further objects and novel features of the present invention will more fully appear from the following detailed description when the same is read in conjunction with the accompanying drawings. It is to be expressly understood, however, that the drawings are for the purpose of illustration only and are not intended as a definition of the limits of the invention.

### BRIEF DESCRIPTION OF THE DRAWINGS

FIG. 1 is a block diagram of one example of a structure of a pulse data detecting apparatus 1 according to a first embodiment of the present invention;

FIG. 2A to FIG. 2F are schematic views each depicting an example of arrangement of light-emitting elements 14-1 to 14-M and light-receiving elements 15-1 to 15N in the pulse data detecting apparatus 1 according to the first embodiment;

FIG. 3 is a first flowchart of the pulse data detecting method performed by the pulse data detecting apparatus 1 according to the first embodiment;

FIG. 4 is a second flowchart of the pulse data detecting method performed by the pulse data detecting apparatus 1 according to the first embodiment;

FIG. 5 is a third flowchart of the pulse data detecting method performed by the pulse data detecting apparatus 1 according to the first embodiment;

FIG. 6 is a flowchart of a pulse data detecting method performed by a pulse data detecting apparatus 1 according to a second embodiment of the present invention;

FIG. 7 is a flowchart of an example of processing of making light emission intensity appropriate, applied to the second embodiment;

FIG. 8 is a flowchart of a specific example when a specific scheme of a method of judging an appropriate combination of a light-receiving element and a light-emitting element is applied to the pulse data detecting method according to the present invention;

FIG. 9 is a flowchart of an example of the method of judging an appropriate combination of a light-receiving element(s) and light-emitting elements applied to a specific example of the pulse data detecting method according to the present invention; and

FIG. 10A and FIG. 10B are diagrams each depicting a first example of measurement data obtained by the pulse data detecting method according to the specific example and analysis data obtained by frequency analysis;

FIG. 11A and FIG. 11B are diagrams each depicting a second example of measurement data obtained by the pulse data detecting method according to the specific example and analysis data obtained by frequency analysis;

FIG. 12A and FIG. 12B are diagrams each depicting a third example of measurement data obtained by the pulse data detecting method according to the specific example and analysis data obtained by frequency analysis; and

FIG. 13 is a flowchart of another example of the method of judging an appropriate combination of a light-receiving element(s) and light-emitting elements applied to a specific example of the pulse data detecting method according to the present invention.

### DETAILED DESCRIPTION OF THE PREFERRED EMBODIMENTS

The pulse data detecting apparatus, pulse data detecting method and pulse data detection program according to the present invention are described in detail below with embodiments. The following description is made in the case where a reflective-type optical pulse data detecting apparatus is applied. Also, when a transmission-type is applied, the apparatus basically has a similar structure and operation.

### A. First Embodiment

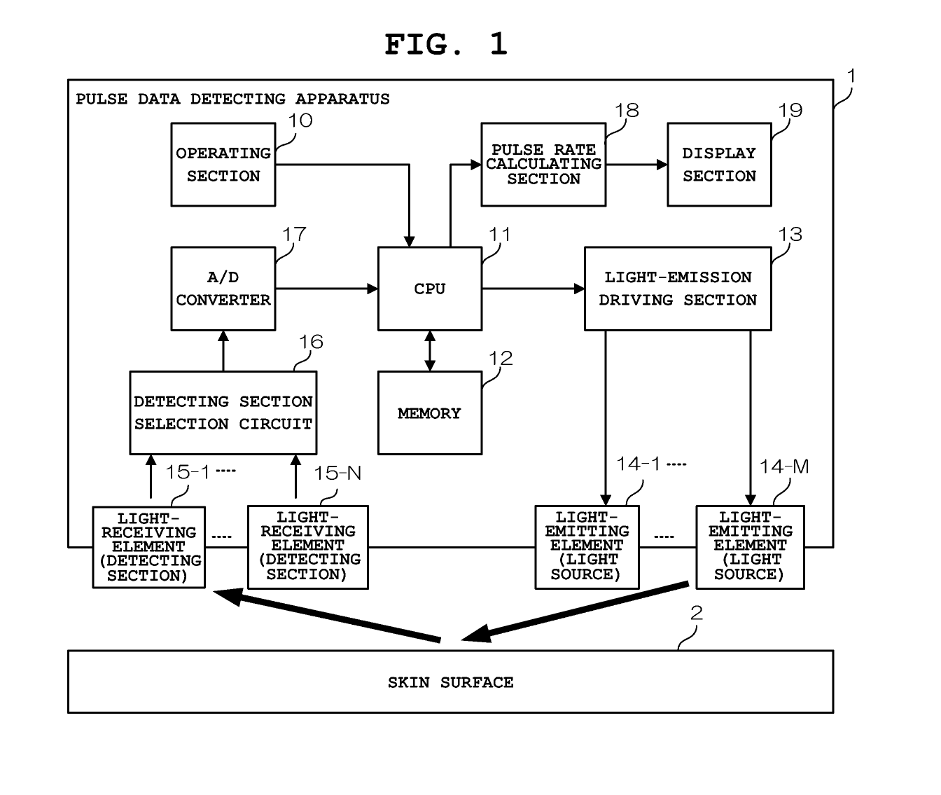

FIG. 1 is a block diagram of one example of a structure of a pulse data detecting apparatus 1 according to a first embodiment of the present invention. In FIG. 1, the pulse data detecting apparatus 1 includes an operating section 10, a CPU 11, a memory 12, a light-emission driving section 13, light-emitting elements (light sources) 14-1 to 14-M, light-receiving elements (detecting sections) 15-1 to 15-N, a detecting section selection circuit 16, an A/D converter 17, a pulse rate calculating section 18, and a display section 19.

The operating section 10 has, for example, a power supply switch operated by a user as a test subject and an operation control switch for controlling the start and stop of a sensing operation.

The CPU 11 performs processing by following a control program stored in the memory 12, and thereby controls pulse measurement, calculation of a pulse rate, and a display operation of the pulse rate. Also, the CPU 11 feeds back to the light-emission driving section 13 based on the detected light amount, and controls, independently or in combination, which light-emission element to light up among the light-emitting elements 14-1 to 14-M, the light emission amount of light-emitting elements to light up, and the number of light-emitting elements to light up, thereby causing the plurality of light-emitting elements 14-1 to 14-M to emit light in a plurality of light-emission patterns. Furthermore, the CPU 11 determines an appropriate combination of a light-emission pattern (light-emitting element) and a light-receiving element satisfying a predetermined condition (an adequate condition) based on an electrical signal (an output signal) outputted from each of the light-receiving elements 15-1 to 15-N when light is emitted in the light-emission pattern described above.

The memory 12 stores measurement data, a control program, data generated at the time of executing the control program, and the like. The light-emission driving section 13 causes a predetermined number of light-emitting elements 14 arranged at predetermined positions among the light-emitting elements (light sources) 14-1 to 14-M to emit light with a predetermined light emission amount, by following the control from the CPU 11.

The light-emitting elements (light sources) 14-1 to 14-M are each made of an LED or the like, and M, at least two (M=2) or more, of the light-emitting elements are arranged on a bottom of a housing (a surface which abuts on a skin surface 2). By following the driving control of the light-emission driving section 13, the light-emitting elements (light sources) 14-1 to 14-M each irradiate the skin surface 2 with a predetermined light emission amount of visible light (for example, green visible light of a wavelength of approximately 525 nm). This reflective-type detecting method using visible light has advantages of being less influenced by reflected light from blood flows in veins and arteries that are present deeply inside the body because of low transmittance of visible light inside the body and of being less influenced by a propagation time lag in heartbeats occurring in each blood vessel due to blood flow path length.

The light-receiving elements (detecting sections) 15-1 to 15-N are each made of an illuminance sensor, a photodiode, or the like, and N, at least one (N=1) or more, of the light-receiving elements are arranged on the bottom of the housing (the surface which abuts on the skin surface 2). The light-receiving elements (detecting sections) 15-1 to 15-N each receive reflected light emitted from any of the light-emitting elements (light sources) 14-1 to 14-M and reflected on the skin surface 2, and output an output signal according to the light reception amount or light reception intensity.

The detecting section selection circuit 16 sequentially selects one light-receiving element 15-i (i=1, 2, . . . , N) from the light-receiving elements (detecting sections) 15-1 to 15-N for each of light emission patterns by the light-emitting elements (light sources) 14-1 to 14-M by following a predetermined condition, and supplies an output signal according to the light amount of the reflected light received by the selected light-receiving element 15-i to the A/D converter 17.

The A/D converter 17 convers the output signal from the light-receiving element 15-i selected by the detecting section selection circuit 16 to digital data (sensor data), and supplies the digital data to the CPU 11. The pulse rate calculating section 18 performs processing by following a predetermined algorithm program, and thereby processes the sensor data obtained from the light-receiving element 15-j in an appropriate combination of the light emission pattern (light-emitting element) and a light-receiving element 15-j (j=1, 2, . . . , N) satisfying a predetermined condition and determined by the CPU 11 to calculate a pulse rate. The pulse rate calculating section 18 may be a computational function incorporated in the CPU 11. Also, the present invention is not limited to the pulse rate and, as will be described further below, various information regarding blood flows included in pulse waveform data (pulse wave data) may be calculated for output.

The display section 19 has a display device such as a liquid-crystal display panel or an organic EL display panel capable of color or monochrome display, displaying the pulse rate calculated by the pulse rate calculating section 18. The display section 19 is not limited thereto. As described above, as pulse data, pulse waves (specifically, pulse waveform data), pitch, and the like may be displayed. For example, pulse waveform data (pulse wave data) includes various information regarding blood flows. That is, the pulse data can be applied as an important parameter for judging health and physical conditions (such as clogging of blood vessels, blood vessel age, and judgment of a tension state), exercise condition, and the like. The display section 19 may display the judgment results by using specific character information, light emission pattern, or the like.

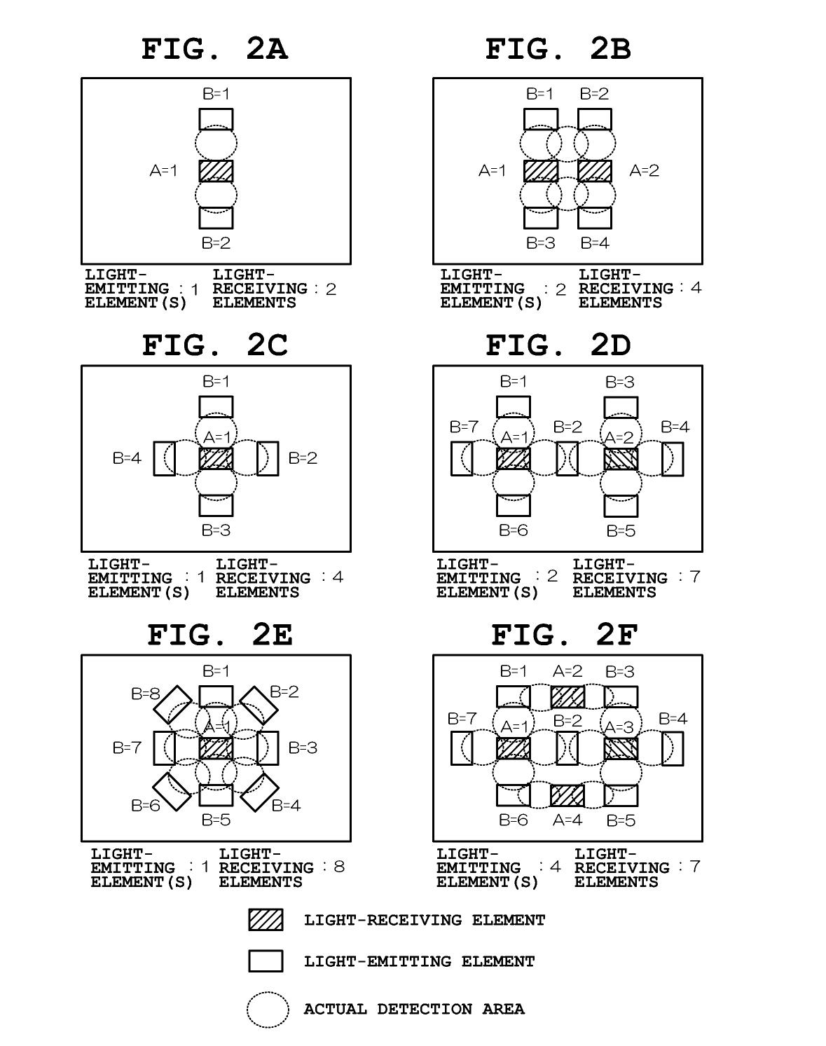

FIG. 2A to FIG. 2F are schematic views each depicting an example of arrangement of light-emitting elements 14-1 to 14-M and light-receiving elements 15-1 to 15N in the pulse data detecting apparatus 1 according to the first embodiment. In FIG. 2A to FIG. 2F, for convenience of illustration, the light-receiving elements 15-1 to 15-N are represented as “A (=1, 2, . . . N)”, and the light-emitting elements 14-1 to 14-M are represented as “B (=1, 2, . . . , M)”.

FIG. 2A depicts an example where one light-receiving element A (=1) is arranged approximately at the center and two light-emitting elements B (=1 and 2) are arranged with a predetermined space apart from each other so as to interpose the light-receiving element A therebetween. FIG. 2B depicts an extended version of the example of arrangement depicted in FIG. 2A, where two light-receiving elements A (=1 and 2) are arranged approximately at the center with a predetermined space apart from each other and four light-emitting elements B (=1, 2, 3, and 4) are arranged with a predetermined space apart from each other so as to interpose the light-receiving elements A (=1 and 2) therebetween.

FIG. 2C depicts an example where one light-receiving element A (=1) is arranged approximately at the center and four light-emitting elements B (=1, 2, 3, and 4) are arranged with a predetermined space apart from each other so as to surround the light-receiving element A from four directions. FIG. 2D depicts an extended version of the example of arrangement depicted in FIG. 2C, where two light-receiving elements A (=1 and 2) are arranged so as to interpose one light-emitting element B (=2) therebetween and seven light-emitting elements B (=1 to 7) including the light-emitting element B (=2) are arranged with a predetermined space apart from each other so as to surround each of the light-receiving elements A (=1 and 2) from four directions.

FIG. 2E depicts an example where one light-receiving element A (=1) is arranged approximately at the center and eight light-emitting elements B (=1 to 8) are arranged so as to surround the light-receiving element A from eight directions. FIG. 2F depicts an extended version of the example of arrangement depicted in FIG. 2D, where one light-receiving element A (=2) is further arranged between two light-emitting elements B (=1 and 3) and one light-receiving element A (=4) is further arranged between two light-emitting elements B (=5 and 6).

That is, in the present embodiment, as depicted in FIG. 2A to FIG. 2F, a plurality of light-emitting elements B are arranged so as to surround or interpose one or more light-receiving elements A. The examples of arrangement of the light-receiving elements A and the light-emitting elements B are merely examples, and the present invention is not limited thereto. In the present invention, for example, the light-receiving elements A may be arranged around the light-emitting elements B. Specifically, the structure may be entirely reversed to the arrangement of the light-receiving elements A and the light-emitting elements B depicted in the drawings. However, in the present invention, since arranging a plurality of light-emitting elements B is a requisite, the structure where only one light-emitting element B is present when the light-receiving elements A and the light-emitting elements B are reversely arranged, as in the examples of arrangement depicted in FIG. 2A, FIG. 2C and FIG. 2D, is excluded.

Furthermore, in the present embodiment, the plurality of light-emitting elements 14-1 to 14-M (B=1, 2, . . . , M) and one or more light-receiving element 15-1 to 15-N (A=1, 2, . . . N) are provided. Depending on the positional relation, absorption light amounts are measured at a plurality of points simultaneously or in a time-division manner. From among the measurement results from the respective points, one or more results of measurement data that are more stable are selected for processing. As a result, pulses can be always stably measured in the present embodiment.

The pulse data detecting apparatus 1 according to the present embodiment can be thought to be of a wristwatch type or wrist band type mounted on the wrist, of an eyeglasses type having a sensor incorporated in a temple portion, or a type of having the earlobe interposed therebetween. Basically, the apparatus may be mounted on any region where human capillary vessels are present. The apparatus may be mounted on an upper arm or a fingertip. Various modes can be thought, such as one wound with a band or one attached on the body surface.

Next, the pulse data detecting method by the pulse data detecting apparatus 1 according to the first embodiment described above is described.

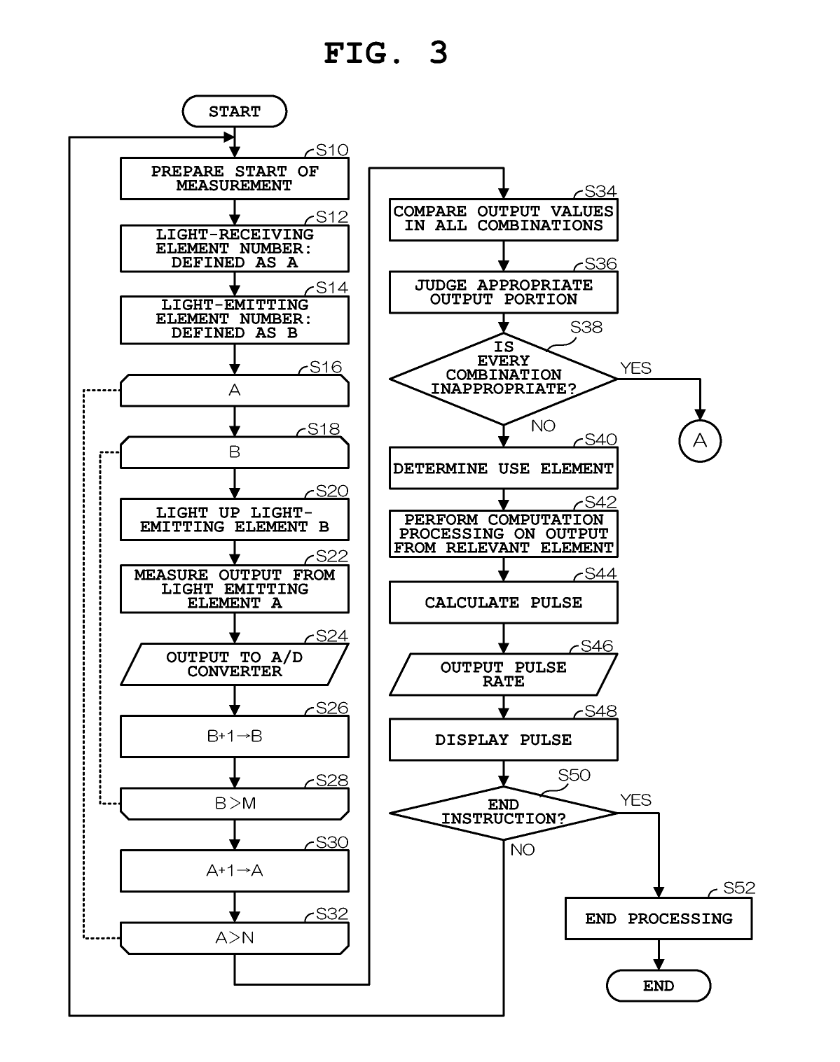

FIG. 3 to FIG. 5 are flowcharts of the pulse data detecting method performed by the pulse data detecting apparatus 1 according to the present embodiment. A user first wears the above-described pulse data detecting apparatus 1 on a measurement region (for example, the wrist or earlobe), and performs a predetermined operation (starts measurement) from the operating section 10. When instructed to start measurement from the user, the CPU 11 performs various processing by following the flowcharts depicted in FIG. 3 to FIG. 5.

First, the CPU 11 performs preparation of starting measurement at Step S10. Next at Step S12, the CPU 11 defines the light-receiving element number as a variable A. The variable A takes any values of 1 to N according to the number of light-receiving elements, and its initial value is 1. Next at Step S14, the CPU 11 defines the light-emitting element number as a variable B. The variable B takes any values of 1 to M according to the number of light-emitting elements, and its initial value is 1. The initial values (=1) of the defined variables A and B are temporarily stored in, for example, the memory 12.

Next, the CPU 11 increments the variable A as the light-receiving element number by 1 to repeat processing from Step S16 to Step S32. In the course of this processing, the CPU 11 increments the variable B as the light-emitting element number by 1 to repeat processing from Step S18 to Step S28. That is, at Step S16 to Step S32, the CPU 11 sequentially performs an operation of driving, for detection, the light-emitting element B and the light-receiving element A in a one-to-one relation for all elements by changing the combination. Details are described below.

First at Step S20, the CPU 11 controls the light-emission driving section 13 to cause the light-emitting element B (=1) to light up. At Step S22, the CPU 11 causes the detecting section selection circuit 16 to select the light-receiving element A (=1) to measure an output from the light-receiving element A (=1). In this measuring operation, the light-emitting element B has its light emission intensity fixed at a specific level (for example, an intermediate level). Next at Step S24, the detecting section selection circuit 16 outputs an output signal from the light-receiving element A (=1) to the A/D converter 17. As a result, the CPU 11 first captures an output value (sensor data) from the light-receiving element A (=1) when the light-emitting element B (=1) is caused to emit light. The CPU 11 associates a combination of the light-receiving element A and the light-emitting element B and the captured output value (sensor data) from the light-receiving element A with each other and temporarily stores the resultant data as measurement data in a predetermined storage area of the memory 12. At this moment, the CPU 11 controls the light-emission driving section 13 to cause the light of the light-emitting element B to be turned off.

Next at Step S26, the CPU 11 increments the variable B by 1 (B+1 to B=2). The incremented variable B is temporarily stored in the memory 12. Then at Step S28, when the variable B is not larger than M indicating a maximum number of light-emitting elements, the CPU 11 returns to Step S18, repeating lighting-up of the light-emitting element B (=2) and measurement of the light-receiving element A (=1). That is, at Step S18 to Step S28, as changing the light-emitting element B to 1, 2, . . . , M, the CPU 11 sequentially captures an output value (sensor data) from the light-receiving element A (=1) and stores the captured values in a predetermined storage area of the memory 12.

Then at Step S28, when the variable B is larger than M indicating the maximum number of light-emitting elements, the CPU 11 increments the variable A by 1 (A+1 to A=2) at Step S30. The incremented variable A is temporarily stored in the memory 12. Then at Step S32, when the variable A is not larger than N indicating a maximum number of light-receiving elements, the CPU 11 returns to Step S16, repeating lighting-up of the light-emitting element B (=1, 2, . . . , M) and measurement of the light-receiving element A (=2) again. That is, as changing the light-emitting element B to 1, 2, . . . , M, the CPU 11 sequentially captures an output value (sensor data) from the light-emitting element A (=2) and stores the captured values in a predetermined storage area of the memory 12.

Thereafter, as described above, as changing the light-emitting element B to 1, 2, . . . , N, the CPU 11 sequentially captures an output value (sensor data) from the light-emitting element A (=1, 2, . . . , N) until the variable B is larger than M indicating the maximum number of light-emitting elements, and thereby obtains output values (sensor data) in all combinations formed of the light-receiving element(s) A and the light-emitting elements B.

Then at Step S32, when the variable A is larger than N indicating the maximum number of light-receiving elements, the CPU 11 compares at Step S34 the output values in all combinations formed of the light-receiving element(s) A and the light-emitting elements B stored in the memory 12. At Step S36, the CPU 11 judges an appropriate output portion. In “judging an appropriate output portion”, based on composite factors such as whether the magnitude of the output level is sufficient and whether the S/N ratio (signal-to-noise ratio) has a value capable of sufficiently extracting a signal, the CPU 11 judges an appropriate combination formed of a light-receiving element(s) A and light-emitting elements B from which an optimum output satisfying a predetermined condition or an appropriate output within a specific range including the optimum output (hereinafter collectively referred to as “appropriate output”) can be obtained. Here, based on whether the output is at least within a specific range set in advance or whether the output satisfies a specific threshold or condition, the CPU 11 judges a combination of a light-receiving element(s) A and the light-emitting elements B from which an appropriate output can be obtained (an appropriate combination). A scheme of judging an appropriate output portion (an appropriate combination judging method) will be described in detail further below.

The CPU 11 then judges at Step S38 whether an appropriate output cannot be obtained from any combination and every combination is inappropriate. Then, when an element combination is present from which an appropriate output that is at least within a specific range set in advance or satisfies a specific threshold or condition can obtained (NO at Step S38), the CPU 11 determines at Step S40 a combination of a light-receiving element(s) A and light-emitting elements B to be used for pulse calculation.

Next at Step S42, the CPU 11 performs computation processing on the output value (sensor data: waveform signal) obtained from the combination of the light-receiving element(s) A and the light-emitting elements B judged as an appropriate output. Furthermore, the pulse rate calculating section 18 calculates a pulse rate (in general, the number of peaks in a waveform for one minute) at Step S44, and outputs the calculated pulse rate to the display section 19 at Step S46. Next at Step S48, the display section 19 displays the calculated pulse rate (numerical value data) as pulse data. The pulse data is not limited to the pulse rate, and measurement of pulse waveform data (pulse wave data) or the like can also be directly applied. Also, the pulse rate calculated at the pulse rate calculating section 18 is associated with the combination, from which an appropriate output is obtained, of the light-receiving element(s) A and the light-emitting elements B, and time data at the time of measurement, etc., and is stored in a predetermined storage area of the memory 12.

Next at Step S50, the CPU 11 judges whether an end instruction is provided to the operating section 10 from the user. When an end instruction is not provided (NO at Step S50), the CPU 11 returns to Step S10, repeating the above-described processing. On the other hand, when an end instruction is provided from the user (YES at Step S50), the CPU 11 performs predetermined end processing (such as storing the pulse rate and discarding measurement data) at Step S52, and then ends the processing.

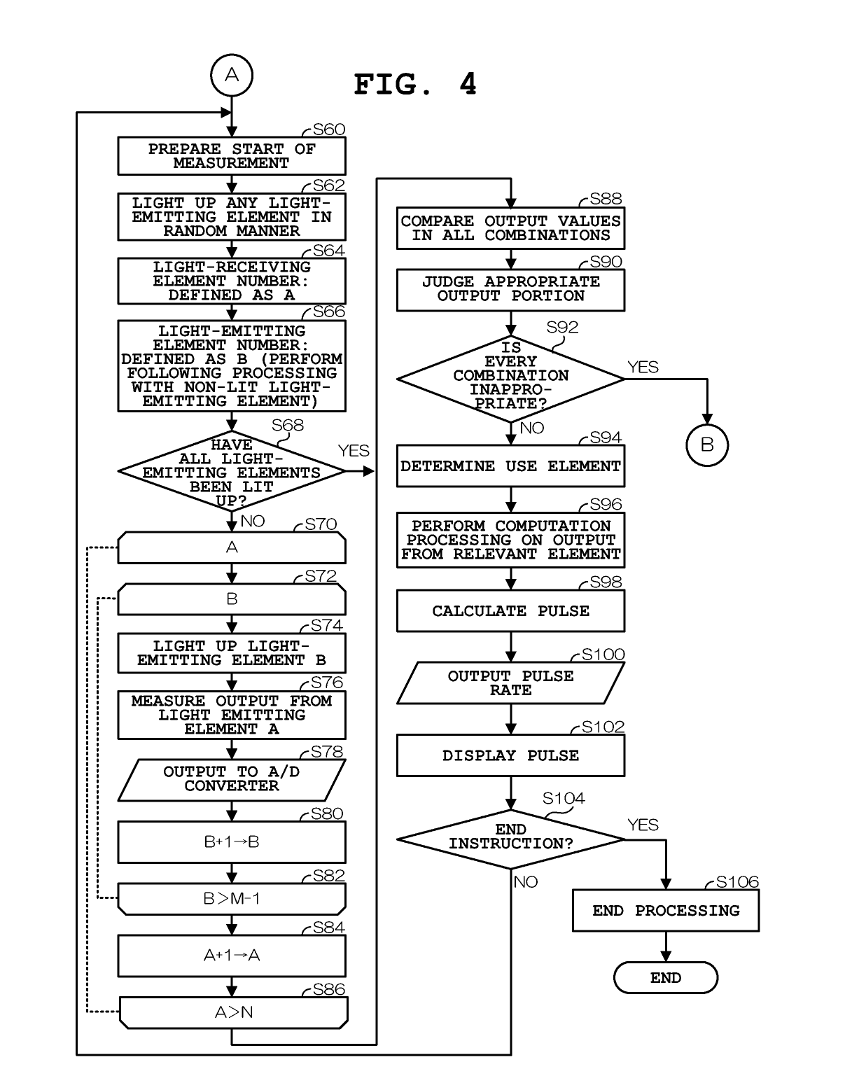

On the other hand, when an element combination is not present where the output is at least within a specific range set in advance or satisfies a specific threshold or condition (YES at Step S38), the CPU 11 proceeds to the flowchart depicted in FIG. 4.

First at Step S60 depicted in FIG. 4, the CPU 11 performs preparation of starting measurement. Next at Step S62, the CPU 11 causes the light-emission driving section 13 to light up any light-emitting element Br in a random manner. Next at Step S64, the CPU 11 defines the light-receiving element number as the variable A. The variable A takes any values of 1 to N according to the number of light-receiving elements, and its initial value is 1. Next at Step S66, the CPU 11 defines the light-emitting element number as the variable B. The variable B takes any values of 1 to M−1 according to the number of light-emitting elements except the light-emitting element Br that lights up in a random manner, and its initial value is 1. The initial values (=1) of the defined variables A and B are temporarily stored in, for example, the memory 12.

Next, as incrementing the variable A as the light-receiving element number by 1, the CPU 11 repeats the processing from Step S70 to Step S86. In the course of this processing, the CPU 11 increments the variable B as the light-emitting element number by 1 to repeat processing from Step S72 to Step S82. That is, at Step S70 to Step S86, the CPU 11 sequentially performs an operation of driving, for detection, a plurality of (two) light-emitting elements formed of one light-emitting element Br selected in a random manner and another one light-emitting element B sequentially specified and one light-receiving element A in a plural (two)-to-one relation for all combinations or any combination by repeating sequential specification and random selection. Details are described below.

First at Step S68, the CPU 11 judges whether all light-emitting elements B (=1, 2, . . . , M) have been lit up. If not all light-emitting elements B have been lit up (NO at Step S68), the CPU 11 lights up the light-emitting element B (=1; except the light-emitting element Br) by controlling the light-emission driving section 13 at Step S74, causes the detecting section selection circuit 16 to select the light-receiving element A (=1) at Step S76, and thereby measures an output from the light-receiving element A (=1). In the measuring operation, the light emission intensity of the light-emitting element B is fixed at a specific level (for example, an intermediate level).

Next at Step S78, the detecting section selection circuit 16 outputs an output signal from the light-receiving element A (=1) to the A/D converter 17. As a result, the CPU 11 captures an output value (sensor data) from the light-receiving element A (=1) when the light-emitting element Br emitting light in a random manner and the light-emitting element B (=1) are lit up. The CPU 11 associates a combination of the light-receiving element A and the light-emitting elements Br and B and the captured output value (sensor data) from the light-receiving element A with each other, and temporarily stores the resultant data as measurement data in a predetermined storage area of the memory 12. At this moment, the CPU 11 controls the light-emission driving section 13 to cause the light of the light-emitting elements B to be turned off.

Next at Step S80, the CPU 11 increments the variable B by 1 (B+1 to B=2). The incremented variable B is temporarily stored in the memory 12. Then at Step S82, when the variable B is not larger than M−1 representing a maximum number of light-emitting elements except the light-emitting element Br, the CPU 11 returns to Step S72, repeating lighting-up of the light-emitting element Br and the light-emitting element B (=2) and measurement of the light-receiving element A (=1). That is, at Step S72 to Step S82, in addition to the light-emitting element Br lit up in a random manner, as changing the light-emitting element B to be lit up to 1, 2, . . . M−1, the CPU 11 sequentially captures an output value (sensor data) from the light-receiving element A (=1) and stores the captured values in a predetermined storage area of the memory 12.

Then at Step S82, when the variable B is larger than M−1 representing the maximum number of light-emitting elements except the light-emitting element Br, the CPU 11 increments the variable A by 1 (A+1 to A=2) at Step S84. Then at Step S86, when the variable A is not larger than N indicating the maximum number of light-receiving elements, the CPU 11 returns to Step S70, repeating lighting-up of the light-emitting element Br and the light-emitting element B (=1, 2, . . . M−1) and measurement of the light-receiving element A (=2). That is, in addition to the light-emitting element Br lit up in a random manner, as changing the light-emitting element B to be lit up to 1, 2, . . . , M−1, the CPU 11 sequentially captures an output value (sensor data) from the light-receiving element A (=2) and stores the captured values in a predetermined storage area of the memory 12.

Thereafter, as described above, as changing the light-emitting element B to 1, 2, . . . , M−1, the CPU 11 sequentially captures output values (sensor data) from the light-emitting element A (=1, 2, . . . , N) until the variable B is larger than M−1 indicating the maximum number of light-emitting elements except the light-emitting element Br, and thereby obtains output values (sensor data) in all combinations each formed of two light-emitting elements and one light-receiving element.

Then at Step S86, when the variable A is larger than N indicating the maximum number of light-receiving elements, the CPU 11 compares at Step S88 the output values in all combinations each formed of two light-emitting elements and one light-receiving element stored in the memory 12. At Step S90, the CPU 11 judges an appropriate output portion. In “judging an appropriate output portion”, as with Step S36 depicted in the flowchart of FIG. 3, based on composite factors such as whether the magnitude of the output level is sufficient and whether the S/N ratio has a value capable of sufficiently extracting a signal, the CPU 11 judges an appropriate combination. Here, the CPU 11 judges an appropriate combination based on whether the output is at least within a specific range set in advance or whether the output satisfies a specific threshold or condition.

The CPU 11 then judges at Step S92 whether an appropriate output cannot be obtained from any combination and every combination is inappropriate. Then, when there is an element combination from which an appropriate output that is at least within a specific range set in advance or satisfies a specific threshold or condition can be obtained (NO at Step S92), the CPU 11 determines at Step S94 a combination of the light-receiving element A, the light-emitting element Br, and the light-emitting element B to be used for pulse calculation.

Next at Step S96, the CPU 11 performs computation processing on the output value (sensor data: waveform signal) obtained from the combination of the light-receiving element A, the light-emitting element Br and the light-emitting element B judged as an appropriate output. Furthermore, the pulse rate calculating section 18 calculates a pulse rate (in general, the number of peaks in a waveform for one minute) at Step S98, and outputs the calculated pulse rate to the display section 19 at Step S100. Next at Step S102, the display section 19 displays the calculated pulse rate (numerical value data) as pulse data. The pulse data is not limited to the pulse rate, and measurement of pulse waveform data (pulse wave data) or the like can also be directly applied. Also, the pulse rate calculated at the pulse rate calculating section 18 is associated with the combination, from which an appropriate output is obtained, of the light-receiving element A, the light-emitting element Br and the light-emitting element B, and time data at the time of measurement, etc., and is stored in a predetermined storage area of the memory 12.

Next at Step S104, the CPU 11 judges whether an end instruction is provided to the operating section 10 from the user. When an end instruction is not provided (NO at Step S104), the CPU 11 returns to Step S60, repeating the above-described processing. In this case, at Step S62, any different light-emitting element Br is lit up in a random manner by the light-emission driving section 13, and the combination of the light-receiving element A, the light-emitting element Br, and the light-emitting element B is changed. Therefore, the output value (sensor data) obtained from the changed combination is also different.

On the other hand, when an end instruction is provided from the user (YES at Step S104), the CPU 11 performs predetermined end processing (such as storing the pulse rate and discarding measurement data) at Step S106, and then ends the processing.

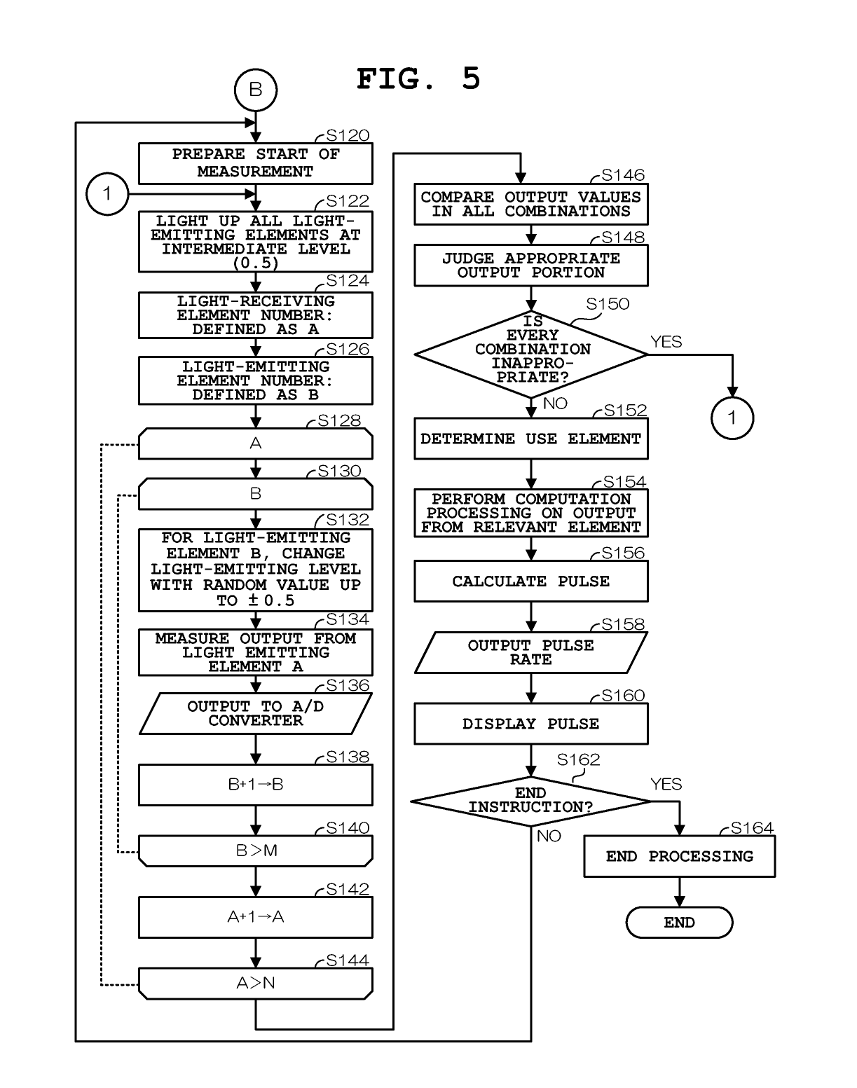

On the other hand, when an element combination is not present where the output is at least within a specific range set in advance or satisfies a specific threshold or condition (YES at Step S92), the CPU 11 proceeds to the flowchart depicted in FIG. 5.

In the flowchart depicted in FIG. 4, two light-emitting elements Br and B are lit up. However, the present invention is not limited thereto, and two or more light-emitting elements may be lit up. Also, in the method described above, for the purpose of reduction in processing time, one of the plurality of light-emitting elements to be lit up is selected in a random manner. However, the present invention is not limited thereto, and light-emitting elements may be sequentially selected regularly or in a pattern based on a specific algorithm. That is, any scheme can be taken as long as a plurality of light-emitting elements are selected (for all combinations or any combination).

First at Step S120 depicted in FIG. 5, the CPU 11 performs preparation of starting measurement. Next at Step S122, the CPU 11 causes the light-emission driving section 13 to light up all light-emitting element B (=1 to M) with light emission intensity at a specific level (for example, an intermediate level; 0.5). Next at Step S124, the CPU 11 defines the light-receiving element number as the variable A. The variable A takes any values of 1 to N according to the number of light-receiving elements, and its initial value is 1. Next at Step S126, the CPU 11 defines the light-emitting element number as the variable B. The variable B takes any values of 1 to M according to the number of light-emitting elements B, and its initial value is 1. The initial values (=1) of the defined variables A and B are temporarily stored in, for example, the memory 12.

Next, as incrementing the variable A as the light-receiving element number by 1, the CPU 11 repeats the processing from Step S128 to Step S144. In the course of this processing, the CPU 11 increments the variable B as the light-emitting element number by 1 to repeat processing from Step S130 to Step S140. That is, at Step S128 to Step S144, with all light-emitting elements Ball being lit up with light emission intensity at a specific level (for example, an intermediate level; 0.5), the CPU 11 sequentially performs an operation of changing (increasing and decreasing) the light emission intensity (light amount) of one light-emitting element B sequentially specified for detection with one light-receiving element A for all light-emitting elements by changing the combination. Details are described below.

First at Step S132, the CPU 11 controls the light-emission driving section 13 to light up the light-emitting element B (=1) by changing the light emission level (light emission amount) with a random number value up to ±0.5. At Step S134, the CPU 11 causes the detecting section selection circuit 16 to select the light-receiving element A (=1), and thereby measures an output from the light-receiving element A (=1).

Next at Step S136, the detecting section selection circuit 16 outputs an output signal from the light-receiving element A (=1) to the A/D converter 17. As a result, in a state where all light-emitting elements Ball are caused to emit light at a specific level (for example, an intermediate level; 0.5), the CPU 11 captures an output value (sensor data) from one light-receiving element A (=1) when the light amount of one light-emitting element B (=1) sequentially specified is changed (increased and decreased) in a random manner. The CPU 11 associates a combination of the light-receiving element A, the light-emitting elements Ball caused to emit light at the specific level and the light-emitting element B with the light amount changed in a random manner, and the captured output value (sensor data) from the light-receiving element A with each other and temporarily stores the resultant data as measurement data in a predetermined storage area of the memory 12. At this moment, the CPU 11 controls the light-emission driving section 13 to cause the light-emitting element B to be back to the original specific level (for example, an intermediate level; 0.5).

Next at Step S138, the CPU 11 increments the variable B by 1 (B+1 to B=2). The incremented variable B is temporarily stored in the memory 12. Then at Step S140, when the variable B is not larger than M indicating the maximum number of light-emitting elements, the CPU 11 returns to Step S130, repeating measurement of one light-receiving element A (=1) when the light amount of one light-emitting element B (=2) sequentially specified is changed (increased and decreased) in a random manner in a state where all light-emitting elements B are caused to emit light at the specific level (for example, an intermediate level: 0.5). That is, at Step S130 to Step S140, in a state where all light-emitting elements Ball are caused to emit light at the specific level (for example, an intermediate level; 0.5), as changing one light-emitting element B sequentially specified to 1, 2, . . . , M, the CPU 11 changes (increases and decreases) the light emission intensity (light amount) in a random manner to sequentially capture an output value (sensor data) from the light-emitting element A (=1) and store the captured values in a predetermined storage area of the memory 12.

Then at Step S140, when the variable B is larger than M indicating the maximum number of light-emitting elements, the CPU 11 increments the variable A by 1 (A+1 to A=2) at Step S142. Then at Step S144, when the variable A is not larger than N indicating the maximum number of light-receiving elements, the CPU 11 returns to Step S128, repeating lighting-up in which the light amount of the light-emitting element B (=1, 2, . . . , M) and measurement of the light-receiving element A (=2) again in a state where all light-emitting elements B are caused to emit light at the specific level (for example, an intermediate level: 0.5). That is, in a state where all light-emitting elements Ball are caused to emit light at the specific level (for example, an intermediate level; 0.5), as changing one light-emitting element B sequentially specified to 1, 2, . . . , M, the CPU 11 changes (increases and decreases) the light amount to sequentially capture an output value (sensor data) from the light-emitting element A (=2) and store the captured values in a predetermined storage area of the memory 12.

Thereafter, as described above, in a state where all light-emitting elements Ball are caused to emit light at the specific level (for example, an intermediate level; 0.5), as changing the light-emitting element B whose light amount is changed in a random manner to 1, 2, . . . , M−1, the CPU 11 sequentially captures an output value (sensor data) from the light-receiving element A (=1, 2, . . . , N). As a result, the CPU 11 obtains output values (sensor data) in all combinations each formed of all light-emitting elements Ball emitting light at a predetermined level, any one of the light-emitting elements B whose light amount is changed in a random manner and one light-receiving element A.

Then at Step S144, when the variable A is larger than N indicating the maximum number of light-receiving elements, the CPU 11 compares the output values in all combinations stored in the memory 12 at Step S146 and judges an appropriate output portion at Step S148. In “judging an appropriate output portion”, as with Step S36 depicted in the flowchart of FIG. 3, based on composite factors such as whether the magnitude of the output level is sufficient and whether the S/N ratio has a value capable of sufficiently extracting a signal, the CPU 11 judges an appropriate combination. Here, the CPU 11 judges an appropriate combination based on whether the output is at least within a specific range set in advance or whether the output satisfies a specific threshold or condition.

The CPU 11 then judges at Step S150 whether an appropriate output cannot be obtained from any combination and every combination is inappropriate. Then, when there is an element combination from which an appropriate output that is at least within a specific range set in advance or satisfies a specific threshold or condition can be obtained (NO at Step S150), the CPU 11 determines at Step S152 a combination of all light-emitting elements Ball emitting light at a predetermined level, any one of the light-emitting elements B whose light amount is changed in a random manner, and one light-receiving element A to be used for pulse calculation.

Next at Step S154, the CPU 11 performs computation processing on the output value (sensor data: waveform signal) obtained from the combination, judged as an appropriate output, of all light-emitting elements Ball emitting light at the predetermined level, any one of the light-emitting elements B whose light amount is changed in a random manner and the light-receiving element A. Furthermore, the pulse rate calculating section 18 calculates a pulse rate (in general, the number of peaks in a waveform for one minute) at Step S156, and outputs the calculated pulse rate to the display section 19 at Step S158. Next at Step S160, the display section 19 displays the calculated pulse rate (numerical value data) as pulse data. The pulse data is not limited to the pulse rate, and measurement of pulse waveform data (pulse wave data) or the like can also be directly applied. Also, the pulse rate calculated at the pulse rate calculating section 18 is associated with the combination, from which an appropriate output is obtained, of the light-receiving element A, all light-emitting elements Ball emitting light at the predetermined level and the light-emitting element B whose light amount is changed in a random manner, and time data at the time of measurement, etc., and is stored in a predetermined storage area of the memory 12.

Next at Step S162, the CPU 11 judges whether an end instruction is provided to the operating section 10 from the user. When an end instruction is not provided (NO at Step S162), the CPU 11 returns to Step S120, repeating the above-described processing. In this case, at Step S132, since the light-emission driving section 13 changes the light emission level of the selected light-emitting element B with the random number value up to ±0.5, the combination of all light-emitting elements Ball emitting light at the predetermined level, any one of the light-emitting elements B whose light amount is changed in a random manner, and one light-receiving element A is changed. Therefore, the output value (sensor data) obtained from the changed combination is also different.

On the other hand, when an end instruction is provided from the user (YES at Step S162), the CPU 11 performs predetermined end processing (such as storing the pulse rate and discarding measurement data) at Step S164, and then ends the processing.

On the other hand, when an element combination is not present where the output is at least within a specific range set in advance or satisfies a specific threshold or condition (YES at Step S150), the CPU 11 returns to Step S122, repeating the above-described processing.

### Modification Examples

Modification examples of the above-described first embodiment are described next.

In the above-described embodiment, when judged at Step S38 of the flowchart depicted in FIG. 3 that every combination formed of a light-receiving element(s) A and light-emitting elements B is inappropriate (YES at Step S38), the processing is performed in the order from the flowchart depicted in FIG. 4 (lighting up a plurality of elements) to the flowchart depicted in FIG. 5 (changing the light amount). This series of processing is merely an example of the pulse data detecting method according to the present invention, and the present invention is not limited thereto. Also with modification examples as described below, it is possible to judge an appropriate combination formed of a light-receiving element(s) and light-emitting elements to output pulse data (measure a pulse).

For example, as a modification example of the above-described first embodiment, the order of the flowchart depicted in FIG. 4 (lighting up a plurality of elements) and flowchart depicted in FIG. 5 (changing the light amount) may be reversed. Alternatively, after the flowchart depicted in FIG. 3, only either one of the flowcharts of FIG. 4 and FIG. 5 may be performed. That is, in another embodiment of the pulse data detecting method according to the present invention, when judged at Step S38 of the flowchart depicted in FIG. 3 that every combination formed of a light-receiving element(s) A and light-emitting elements B is inappropriate, processing is performed in the order from the flowchart depicted in FIG. 5 (changing the light amount) to the flowchart depicted in FIG. 4 (lighting up a plurality of elements). Furthermore, in still another embodiment, only the processing of the flowchart of FIG. 4 is performed after the flowchart depicted in FIG. 3, or only the processing of the flowchart of FIG. 5 is performed after the flowchart depicted in FIG. 3.

As another modification example of the above-described first embodiment, the flowchart depicted in FIG. 4 (lighting up a plurality of elements) and the flowchart depicted in FIG. 5 (changing the light amount) may be performed without performing the flowchart depicted in FIG. 3, or only either one of the flowchart depicted in FIG. 4 (lighting up a plurality of elements) and the flowchart depicted in FIG. 5 (changing the light amount) may be performed. That is, in still another embodiment of the pulse data detecting method according to the present invention, only the processing of the flowchart depicted in FIG. 4 (lighting up a plurality of elements) and the flowchart depicted in FIG. 5 (changing the light amount) is provided, and the processing is performed in the order from the flowchart depicted in FIG. 4 (lighting up a plurality of elements) to the flowchart depicted in FIG. 5 (changing the light amount) or in the order from the flowchart depicted in FIG. 5 (changing the light amount) to the flowchart depicted in FIG. 4 (lighting up a plurality of elements). In yet still another embodiment, only the processing of the flowchart depicted in FIG. 4 (lighting up a plurality of elements) or only the processing of the flowchart depicted in FIG. 5 (changing the light amount) is performed.

In the above-described embodiment, description of a specific outer structure of the pulse data detecting apparatus 1 is omitted. In general, the light-emitting elements and the light-receiving element are mounted on a circuit board. Originally, pulse measurement can be performed with this structure as it is. However, in addition to reflection from the body surface, direct light due to wrapping from an element side surface may have extremely large influence. For the purpose of eliminating direct light, in the present embodiment, the structure with a light-shielding block arranged around each of the light-emitting elements 14-1 to 14-M and the light-receiving elements 15-1 to 15-N may be applied. As the light-shielding block, a component formed of black resin or the like can be applied.

(Comparison and Verification)

Next, operations and effects of the pulse data measuring apparatus according to the present embodiment is verified and described by taking the pulse data measuring apparatus (a laser blood flow meter) with the structure as in the above-described Related Art section as a comparison target.

In the case of the pulse data measuring apparatus as a comparison target, an area as a pulse measurement target is a certain area present in a position approximately at the center (an intermediate portion) between the arranged positions of the light-emitting elements and the light-receiving element. Therefore, in another portion, measurement cannot be performed unless the pulse data measuring apparatus itself is moved. Accordingly, for example, if an obstacle such as a lentigo is present in the area present in an intermediate portion between the light-emitting elements and the light-receiving element, if capillary vessels are distributed very sparsely, or if body hair distribution is concentrated or is accidentally interposed, stable pulse measurement cannot be performed.

In these cases, depending on the shape or structure of the pulse data measuring apparatus, the placement location can be changed again. However, even if the apparatus is placed again, stable measurement is not necessarily ensured. Accordingly, the user may feel somewhat stress. If the apparatus cannot be placed except in a specific region due to the shape, structure, or the like of the pulse data measuring apparatus, the user falls into a situation where pulse measurement by using the pulse data measuring apparatus cannot be made.

By contrast, in the present embodiment, the plurality of light-emitting elements 14-1 to 14-M are arranged so as to surround one or more light-receiving elements 15-1 to 15-N and, by switching the light emission pattern (the number, the positions, and the light emission amounts of light-emitting elements to emit light) of the light-emitting elements 14-1 to 14-M to emit light, a plurality of points can be measured simultaneously. Here, an equivalent effect can also be obtained in the structure where the plurality of light-receiving elements 15-1 to 15-N are arranged so as to surround the plurality of light-emitting elements 14-1 to 14-M arranged at a center portion.

Also in the present embodiment, regarding light emission timing of the light-emitting elements 14-1 to 14-M, by causing all light-emitting elements 14-1 to 14-M to emit light simultaneously, more intense reflected light can be detected. Alternatively, by sequentially lighting up the plurality of light-emitting elements 14-1 to 14-M, an appropriate measurement range can be selected. As such, according to the present embodiment, a measurable area can be greatly widened without at least moving or remounting the pulse data detecting apparatus, and the possibility of stable pulse measurement is significantly enhanced.

As such, according to the present embodiment, the light emission amounts of the plurality of light-emitting elements are controlled. Therefore, a wide range can be taken as a measurement area regardless of the state of placement of the pulse data detecting apparatus 1 on the human body, and thereby stable pulse measurement can be performed.

In particular, according to the present embodiment, by controlling the light emission amount of the plurality of light-emitting elements, the plurality of light-emitting elements can be caused to emit light in a plurality of light emission patterns. Therefore, a wide range can be taken as a measurement area regardless of the state of placement of the pulse data detecting apparatus 1 on the human body, and thereby stable pulse measurement can be performed.

Here, in the present embodiment, among the plurality of light-emitting elements, the number of light-emitting elements to be lit up, the position of each light-emitting element to be lit up, or the light emission amount of each light-emitting element to be lit up is controlled independently or in combination. As a result, the plurality of light-emitting elements can be caused to emit light in various light emission patterns.

Furthermore, in the present embodiment, among the plurality of light-emitting elements, every time at least two or more different light-emitting elements are combined to sequentially light up simultaneously, an appropriate combination of at least two or more light-emitting elements and a light-receiving element(s) satisfying a predetermined condition is determined based on an electrical signal outputted from the light-receiving element. As a result, various combinations can be achieved and appropriate pulse measurement can be performed.

Still further, in the present embodiment, every time any one of the plurality of light-emitting elements is lit up sequentially, an appropriate combination of any one of the light-emitting elements and the light-receiving element satisfying a predetermined condition is determined based on an electrical signal outputted from the light-receiving element. Then, if an appropriate combination cannot be determined, every time at least two or more different light-emitting elements are combined to sequentially light up simultaneously, an appropriate combination of at least two or more light-emitting elements and a light-receiving element(s) satisfying a predetermined condition is determined based on an electrical signal outputted from the light-receiving element. As a result, the processing can make a transition to more complex control in a stepwise manner, various combinations can be achieved according to the situation at the time of measurement, whereby appropriate pulse measurement can performed.

Yet still further, in the present embodiment, if an appropriate combination of at least two or more light-emitting elements and a light-receiving element(s) cannot be determined, every time at least two or more different light-emitting elements are combined to sequentially light up simultaneously with different light amounts, an appropriate combination of at least two or more light-emitting elements and a light-receiving element(s) satisfying a predetermined condition is determined based on an electrical signal outputted from the light-receiving element. As a result, the processing can make a transition to more complex control in a stepwise manner, various combinations can be achieved according to the situation at the time of measurement, whereby appropriate pulse measurement can performed.

Yet still further, in the present embodiment, every time any one of the plurality of light-emitting elements is lit up sequentially, an appropriate combination of any one of the light-emitting elements and a light-receiving element(s) satisfying a predetermined condition is determined based on an electrical signal outputted from the light-receiving element. Then, if an appropriate combination cannot be determined, every time at least two or more different light-emitting elements are combined to sequentially light up simultaneously with different light amounts, an appropriate combination of at least two or more light-emitting elements and a light-receiving element(s) satisfying a predetermined condition is determined based on an electrical signal outputted from the light-receiving element. As a result, the processing can make a transition to more complex control in a stepwise manner and various combinations can be achieved according to the situation at the time of measurement, whereby appropriate pulse measurement can performed.

Yet still further, according to the present embodiment, the plurality of light-emitting elements are arranged to surround the light-receiving element. Therefore, various combinations of light-emitting elements and a light-receiving element(s) can be achieved with a simple structure.

Yet still further, according to the present embodiment, the number of light-receiving element is at least one. Therefore, various combinations of light-emitting elements and a light-receiving element(s) can be achieved with a simple structure.

Yet still further, according to the present embodiment, a plurality of light-receiving elements are arranged to surround the plurality of light-emitting elements. Therefore, various combinations of light-emitting elements and a light-receiving element(s) can be achieved.

Yet still further, according to the present embodiment, any one of the plurality of light-receiving elements is sequentially selected, and an appropriate combination of a plurality of light emission patterns and any one of the light-receiving elements satisfying a predetermined condition is determined based on an electrical signal outputted from the any one of the light-receiving elements sequentially selected. As a result, various combinations can be achieved according to the situation at the time of measurement, whereby appropriate pulse measurement can performed.

### B. Second Embodiment

Next, a second embodiment according to the present invention is described.

A pulse data detecting apparatus 1 according to the second embodiment has a structure similar to that of the above-described first embodiment (refer to FIG. 1 and FIG. 2A to FIG. 2F), and therefore the structure is not described herein. In the second embodiment, after determining an appropriate combination of light-emitting elements and a light-receiving element(s) satisfying a predetermined condition with the pulse data detecting method of the above-described first embodiment, the CPU 11 controls the light emission intensity of the light-emitting elements at a lowest value capable of appropriate pulse measurement (processing of making light emission intensity appropriate).

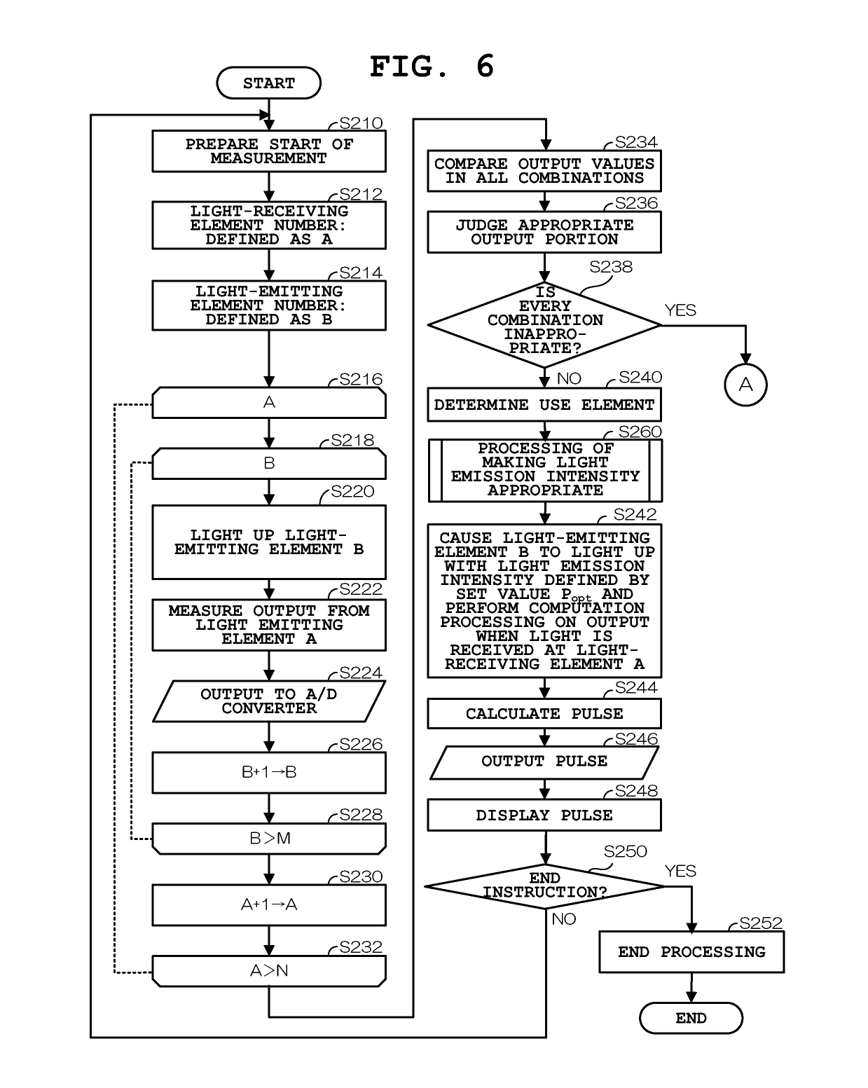

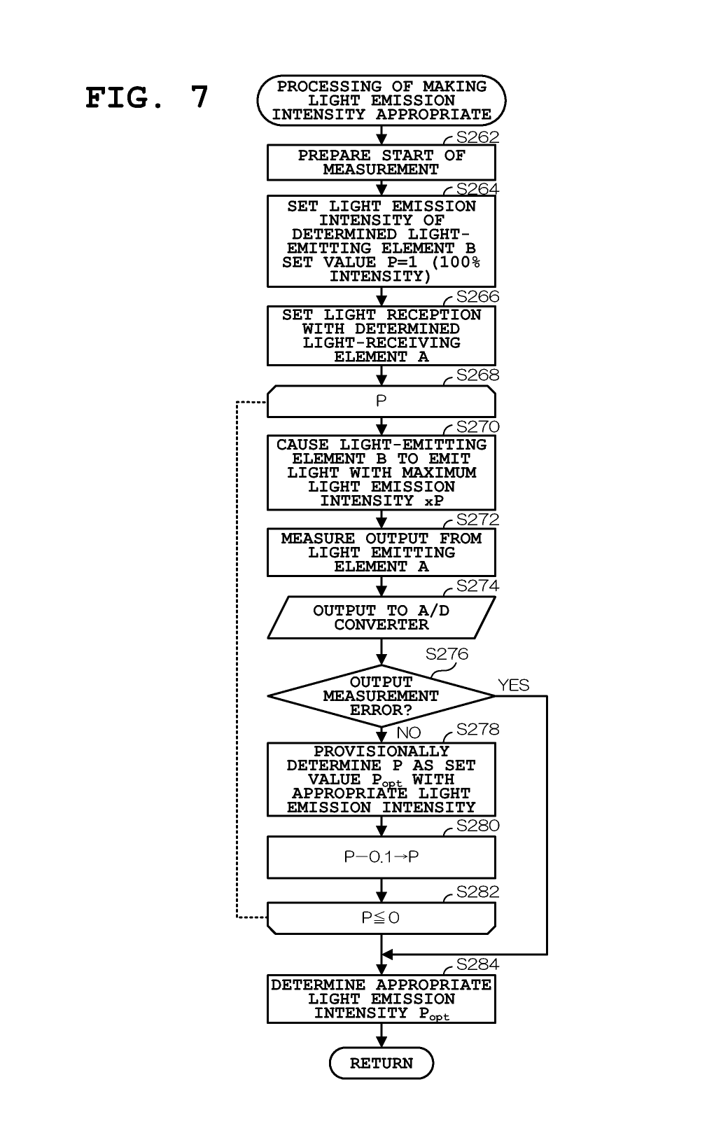

FIG. 6 is a flowchart of a pulse data detecting method performed by a pulse data detecting apparatus 1 according to a second embodiment of the present invention. Here, the case is described where processing of making light emission intensity appropriate according to the present embodiment is applied to the pulse data detecting method depicted in the flowchart of FIG. 3 in the first embodiment. Note that processing procedures identical to those of the flowchart (FIG. 3) in the above-described first embodiment are provided with the same reference numeral. FIG. 7 is a flowchart of an example of processing of making light emission intensity appropriate, applied to the second embodiment.

In the pulse data detecting method according to the present embodiment, a user first wears the pulse data detecting apparatus 1 on a measurement region (for example, the wrist or earlobe), and performs a predetermined operation (starts measurement) from the operating section 10. When an instruction for starting measurement is provided from the user, the CPU 11 performs various processing by following the flowchart depicted in FIG. 6.

Here, a series of processing at Step S210 to Step S240 in the present embodiment correspond to the processing at Step S10 to Step S40 depicted in the flowchart of FIG. 3 in the above-described first embodiment. That is, at Step S210 to Step S240, the CPU 11 performs preparation of starting measurement, and defines the light-receiving element number as the variable A and the light-emitting element number as the variable B. Next, in the series of processing at Step S216 to Step S232, the CPU 11 increments the variable A as the light-receiving element number and the variable B as the light-emitting element number by 1, and thereby sequentially performs an operation of driving, for detection, the light-emitting element B and the light-receiving element A in a one-to-one relation for all elements by changing combinations. In this series of processing, the CPU 11 associates combinations of the light-receiving element(s) A and the light-emitting elements B and the output values (sensor data) from the light-receiving element(s) A in respective combinations with each other, and temporarily stores the resultant data as measurement data in a predetermined storage area of the memory 12.

Next at Step S234, the CPU 11 compares the obtained output values in all combinations each formed of a light-receiving element(s) A and light-emitting elements B. At Step S236, the CPU 11 judges an appropriate output portion. Then at Step S238, when judging that an appropriate output cannot be obtained from any combination and every combination is inappropriate (YES at Step S238), the CPU 11 performs the series of processing of the flowchart depicted in FIG. 4 of the above-described first embodiment. On the other hand, when judging that a combination from which an appropriate output can be obtained is present (NO at Step S238), the CPU determines at Step S240 a combination of a light-receiving element(s) A and light-emitting elements B to be used for pulse calculation.

Next, the CPU 11 performs processing of making light emission intensity appropriate for the light-receiving element A and the light-emitting element B determined to be used for pulse calculation. Here, the CPU 11 follows a flowchart depicted in FIG. 7 to perform a series of processing for setting the light emission intensity of the light-emitting element B determined to be used for pulse calculation at a minimum intensity capable of appropriate pulse measurement.

Specifically, in the processing of making light emission intensity appropriate applied to the present embodiment. The CPU 11 first performs preparation of starting measurement at Step S262. At Step S264, the CPU 11 causes the light-emission driving section 13 to set a set value P defining the light emission intensity of the determined light-emitting element B at an initial value (=1). Here, the light emission intensity defined by the set value P having the initial value (=1) is set at a maximum level of light emission intensity (100% intensity) in the light-emitting element B. That is, in the present embodiment, the light emission intensity of the light-emitting element B is set at a level equal to or lower than the maximum level of light emission intensity by multiplying the maximum level of light emission intensity by the set value P equal to or lower than 1. The light emission intensity defined by the set value P having the initial value (=1) is not limited to the maximum level (100% intensity) in the light-emitting element B, but may be set at, for example, any high level of light emission intensity (for example, 80% intensity). The set value P set herein is temporarily stored in, for example, the memory 12. Next at Step S266, the CPU 11 causes the detecting section selection circuit 16 to perform light-receiving setting for measuring an output from the determined light-receiving element A.

Next, as decrementing the set value P defining the light emission intensity of the light-emitting element B by 0.1, the CPU 11 repeats processing from Step S268 to Step S282. That is, at Step S268 to Step S282, the CPU 11 sequentially performs an operation of driving, for detection, the light-emitting element B and the light-receiving element A determined to be used for pulse calculation in a one-to-one relation as decreasing the light emission intensity of the light-emitting element B. Details are described below.

First at Step S270, the CPU 11 controls the light-emission driving section 13 to cause the light-emitting element B to light up with a light emission intensity defined by (maximum level)×(set value P=1). At Step S272, the CPU 11 causes the detecting section selection circuit 16 to select the light-receiving element A and measures an output therefrom. Next at Step S274, the detecting section selection circuit 16 outputs an output signal from the light-receiving element A to the A/D converter 17. As a result, the CPU 11 first captures the output value (sensor data) from the light-receiving element A when the light-emitting element B is caused to emit light at a maximum level of light emission intensity (100% intensity). The CPU 11 associates the set value P at this moment (that is, the light emission intensity of the light-emitting element B) and the captured output value (sensor data) from the light-receiving element A with each other and temporarily stores the resultant data as measurement data in a predetermined storage area of the memory 12. Also at this moment, the CPU 11 controls the light-emission driving section 13 to cause lighting of the light-emitting element B to be turned off.

Here, at Step S276, the CPU 11 judges whether an error is present in the processing of measuring the captured output value (sensor data) from the light-receiving element A (or whether the output value is adequate). When an error is present in measurement of the output from the light-receiving element A (YES at Step S276), the CPU 11 performs processing at Step S284 onward, which will be described further below. On the other hand, when an error is not present in measurement of the output from the light-receiving element A (NO at Step S276), the CPU 11 judges at Step S278 that the set value P is a set value defining light emission intensity from which an appropriate output value can be obtained, and provisionally determines the set value P as an appropriate set value Pₒₚₜ. The CPU 11 associates the set value P at this moment (appropriate set value Pₒₚₜ) and the captured output value (sensor data) from the light-receiving element A with each other and temporarily stores the resultant data in a predetermined storage area of the memory 12.

Next, at Step S280, the CPU 11 decrements the set value P by 0.1 (P-0.1 to P). The decremented set value P is temporarily stored in, for example, the memory 12. Then at Step S282, when the set value P is not equal to or lower than a set value 0 defining a non-light-emission state, the CPU 11 returns to Step S268, repeating lighting-up of the light-emitting element B with the light emission intensity defined by the decremented set value P (maximum level×P) and the measurement of the light-receiving element A. By repeatedly performing this series of processing for every light emission intensity (that is, every set value P), the latest and lowest set value P defining light emission intensity from which an appropriate output value can be obtained is provisionally determined sequentially as the appropriate set value Pₒₚₜ, and is stored for update in the memory 12.

Then, when the set value P is equal to or lower than the set value 0 defining a non-light-emission state at Step S282 or when judged at Step S276 that an error is present in the processing of measuring the output value from the light-receiving element A (or the output value is inadequate), the CPU 11 determines at Step S284 the latest (current) set value P provisionally determined as the appropriate set value Pₒₚₜ and stored in the memory 12 as a most appropriate set value Pₒₚₜ. The determined appropriate set value Pₒₚₜ is stored in a predetermined area of the memory 12. Thereafter, in the flowchart of FIG. 6, the CPU 11 performs processing at Step S242 onward. Here, a series of processing at Step S242 to Step S252 in the present embodiment correspond to the processing at Step S42 to Step S52 of the above-described first embodiment.

That is, at Step S242, the CPU 11 causes the light-emitting element B to light up with light emission intensity defined by the determined appropriate set value Pt, and performs computation processing on the output value (sensor data) when light is received at the light-receiving element A. Furthermore, the pulse rate calculating section 18 calculates a pulse rate at Step S244, and outputs the calculated pulse rate to the display section 19 at Step S246. The calculated pulse rate is associated with the set value P at that moment (appropriate set value Pₒₚₜ), and time data at the time of measurement, etc., and is stored in a predetermined storage area of the memory 12. Next, at Step S248, the display section 19 displays the calculated pulse rate as pulse data.

Next, at Step S250, the CPU 11 judges whether an end instruction is provided to the operation section 10 from the user. When an end instruction is not provided (NO at Step S250), the CPU 11 returns to Step S210, repeating the above-described pulse rate calculation processing. On the other hand, when an end instruction is provided from the user (YES at Step S250), the CPU 11 performs predetermined end processing (such as storing the pulse rate and discarding measurement data) at Step S252, and then ends the processing.

As such, in the present embodiment, after determining a combination of light-emitting element(s) and a light-receiving element(s) from which an appropriate output can be obtained in the above-described first embodiment, processing of making light emission intensity appropriate for setting lower light emission intensity is performed, in which favorable pulse measurement in this combination can be achieved. As a result, according to the present embodiment, in an appropriate combination of light-emitting elements and a light-receiving element(s), the light emission intensity of the light-emitting elements can be set lower. Therefore, it is possible to provide a pulse data detecting apparatus capable of stable and reliable pulse measurement with small power consumption.

In the present embodiment, the case is described where the processing of making light emission intensity appropriate is applied to the series of processing depicted in the flowchart of FIG. 3 of the pulse data detecting method in the above-described first embodiment. However, the present invention is not limited thereto. That is, the processing of making light emission intensity appropriate applied to the present invention can be any as long as the processing can achieve favorable pulse measurement with lower light emission intensity in a combination, from which an appropriate output can be obtained, of light-emitting elements and light-receiving elements determined by the pulse data detecting method according to the present invention. Therefore, after determining an appropriate combination of light-emitting elements and the light-receiving element(s) by the series of processing depicted in the flowchart of FIG. 4 or FIG. 5 in the above-described first embodiment, the series of processing of making light emission intensity appropriate depicted in the flowchart of FIG. 7 may be performed.

Also, in the above-described first and second embodiments, the pulse measurement period and measurement time are arbitrarily set according to the use purpose of the pulse data, measurement accuracy, and the like. In general, the measurement time is set, for example, on the order of ten to fifteen seconds, or several seconds to one minute depending on the measurement state.

### C. Specific Example of Pulse Data Detecting Method

Next, description is made to a method of judging an appropriate combination of light-receiving element(s) A and light-emitting elements B applied to the pulse data detecting method according to the above-described first and second embodiments.

In the above-described first and second embodiments, it is described that an appropriate output satisfying a predetermined condition can be obtained by the series of processing according to the pulse data detecting method (refer to the flowcharts depicted in FIG. 3 to FIG. 6). Here, a method for judging “an appropriate output satisfying a predetermined condition” and a method for determining a combination (an appropriate combination) of the light-receiving element A and the light-emitting element B from which the appropriate output can be obtained are described, which are both applied to the above-described pulse data detecting method, in detail by using a specific scheme. In the following description, the appropriate output judging method and the appropriate combination determining method are collectively referred to as an “appropriate combination judging method” for convenience.

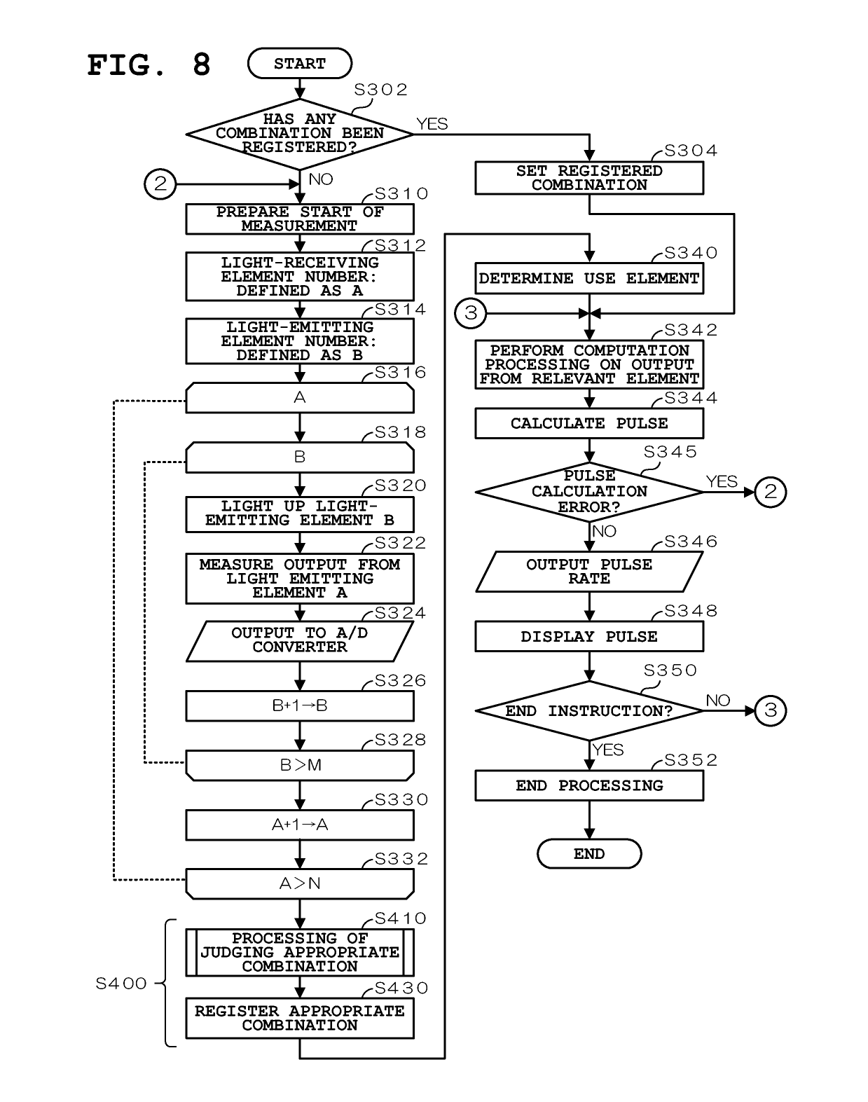

FIG. 8 is a flowchart of a specific example when a specific scheme of a method of judging an appropriate combination of a light-receiving element(s) and light-emitting elements is applied to the pulse data detecting method according to the present invention. Here, the case is described where a specific scheme of the appropriate combination judging method is applied to the pulse data detecting method depicted in the flowchart of FIG. 3 in the above-described first embodiment. Note that processing procedures identical to those of the flowchart (FIG. 3) in the above-described first embodiment are provided with the same reference numeral.

In the pulse data detecting method according to the present specific example, the user first wears the pulse data detecting apparatus 1 on a measurement region (for example, the wrist or earlobe), and performs a predetermined operation (starts measurement) from the operating section 10. When instructed to start measurement from the user, the CPU 11 performs various processing by following the flowchart depicted in FIG. 8.

First, at Step S302, the CPU 11 judges whether a combination of a light-receiving element(s) A and light-emitting elements B has been registered in advance in the memory 12. Here, as the combination registered in the memory 12, for example, a combination judged by a series of processing, which will be described further below, as the latest, most appropriate combination can be applied. Then at Step S302, if a combination of a light-receiving element(s) A and light-emitting elements B has been registered in the memory 12 (YES at Step S302), the CPU 11 reads out the combination from the memory 12, sets the read out combination as an element combination to be used for pulse calculation at Step S304, and performs processing at Step S342 onward, which will be described further below.

On the other hand, at Step S302, if a combination of a light-receiving element(s) A and light-emitting elements B has not been registered in the memory 12 (or a combination has been registered but is not the most appropriate combination; No at Step S302), as with the case of the above-described first embodiment, the following series of processing at Step S310 to S332 are performed. Here, the series of processing at Step S310 to S332 correspond to Steps S10 to Step S32 depicted in the flowchart of FIG. 3 of the first embodiment.

That is, at Step S310 to Step S332, the CPU 11 performs preparation of starting measurement, and defines the light-receiving element number as the variable A and the light-emitting element number as the variable B. Next, in the series of processing at Step S316 to Step S332, the CPU 11 increments the variable A as the light-receiving element number and the variable B as the light-emitting element number by 1, and thereby sequentially performs an operation of driving, for detection, the light-emitting element B and the light-receiving element A in a one-to-one relation for all elements by changing combinations. In this series of processing, the CPU 11 associates combinations of the light-receiving element(s) A and the light-emitting elements B and the output values (sensor data) from the light-receiving element A in respective combinations with each other and temporarily stores the resultant data as measurement data in a predetermined storage area of the memory 12. Here, the operation of measuring and capturing an output from the light-receiving element A at Step S322 and S324 continues for a predetermined time (for example, on the order of several seconds to one minute, preferably several tens of seconds or more), during which measurement data including a predetermined number of pulses (for example, five to forty-five pulses, preferable several tens of pulses or more) is obtained and is stored in the memory 12.

Next at Step S400, the CPU 11 judges an appropriate combination of a light-receiving element(s) A and light-emitting elements B. Specifically, the CPU 11 applies a frequency analysis scheme by Fourier transform described below to perform processing of judging an appropriate combination of a light-receiving element(s) and light-emitting elements (Step S410) and processing of registering the judged appropriate combination (Step S430).

(First Scheme)

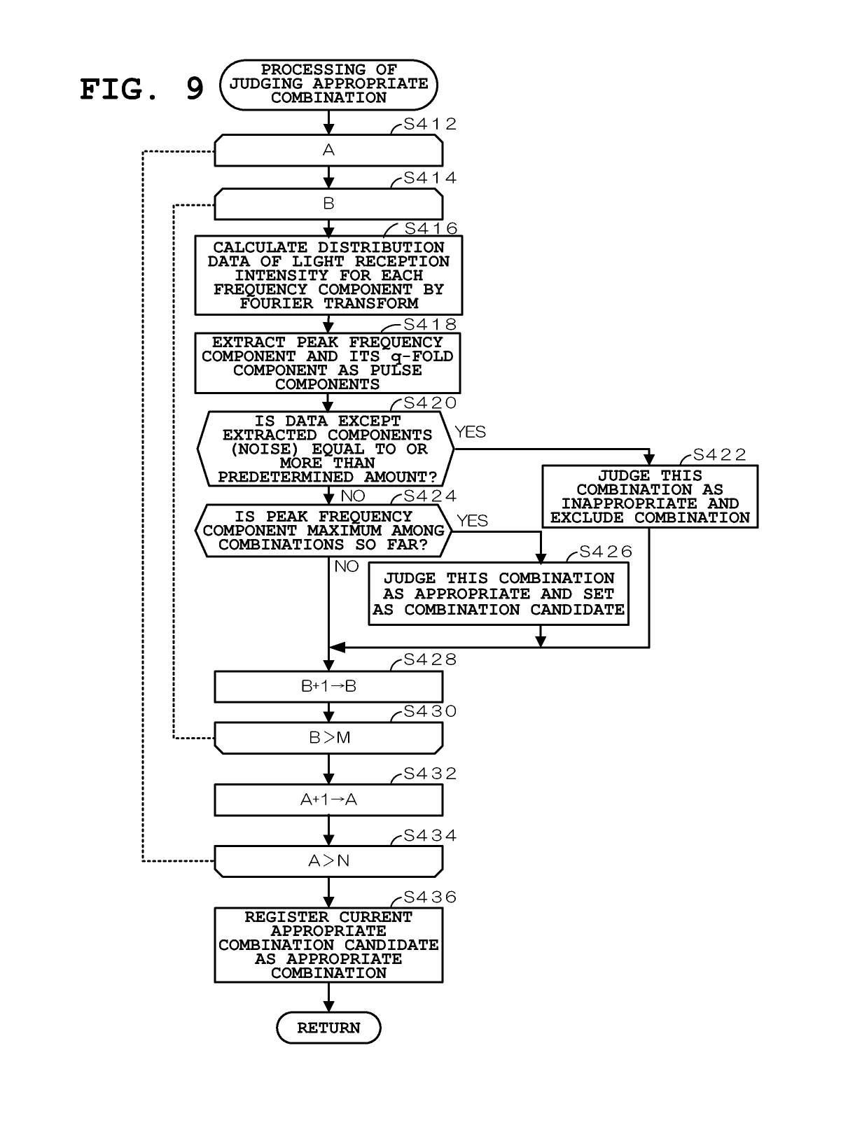

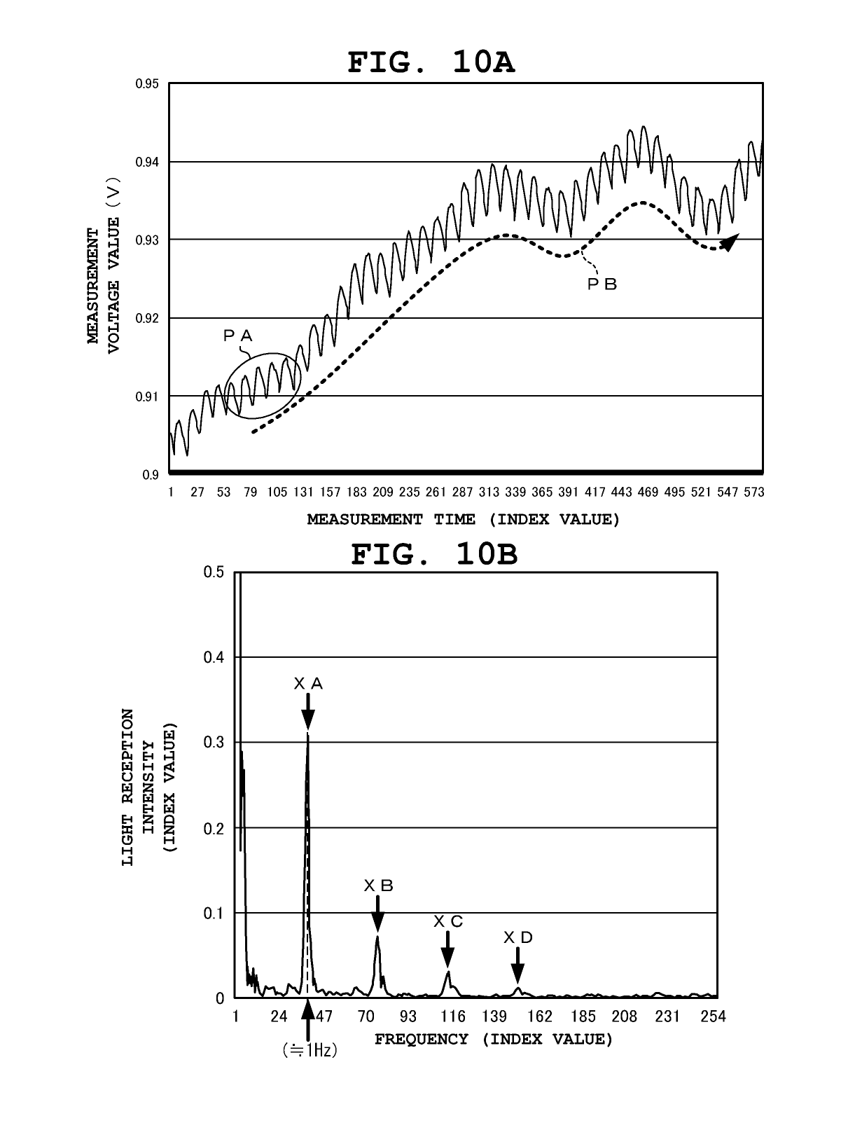

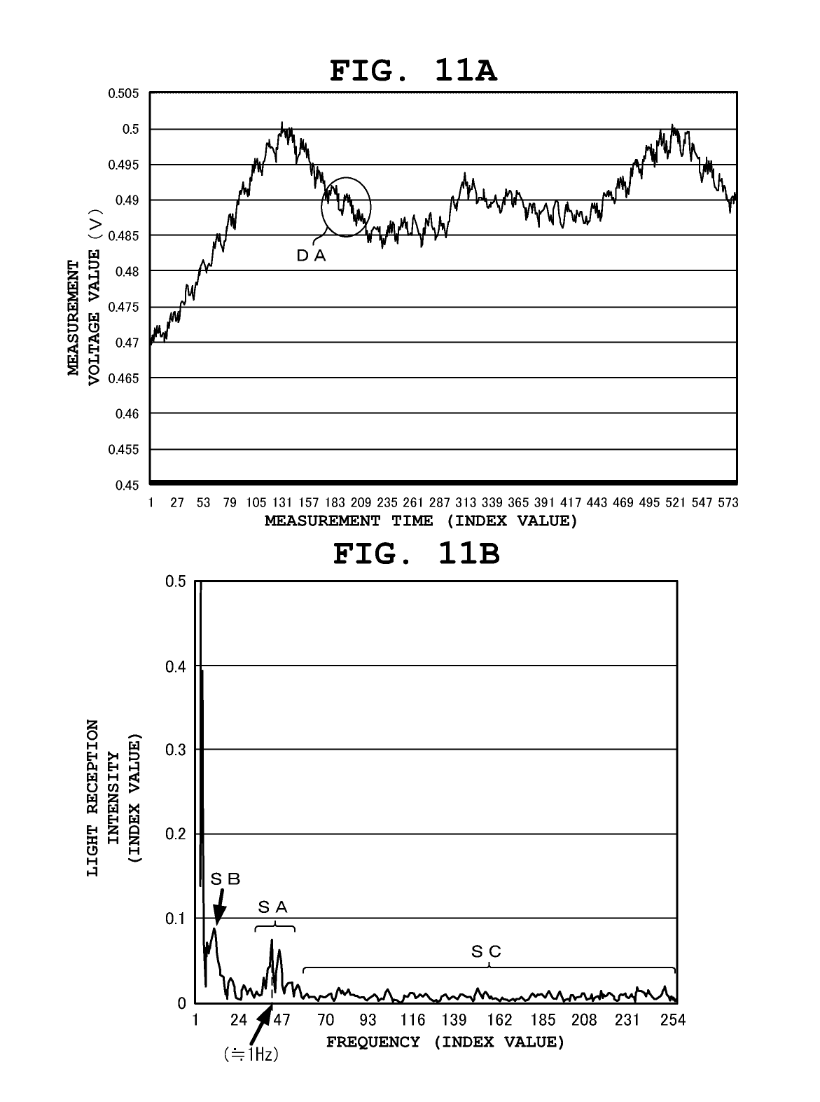

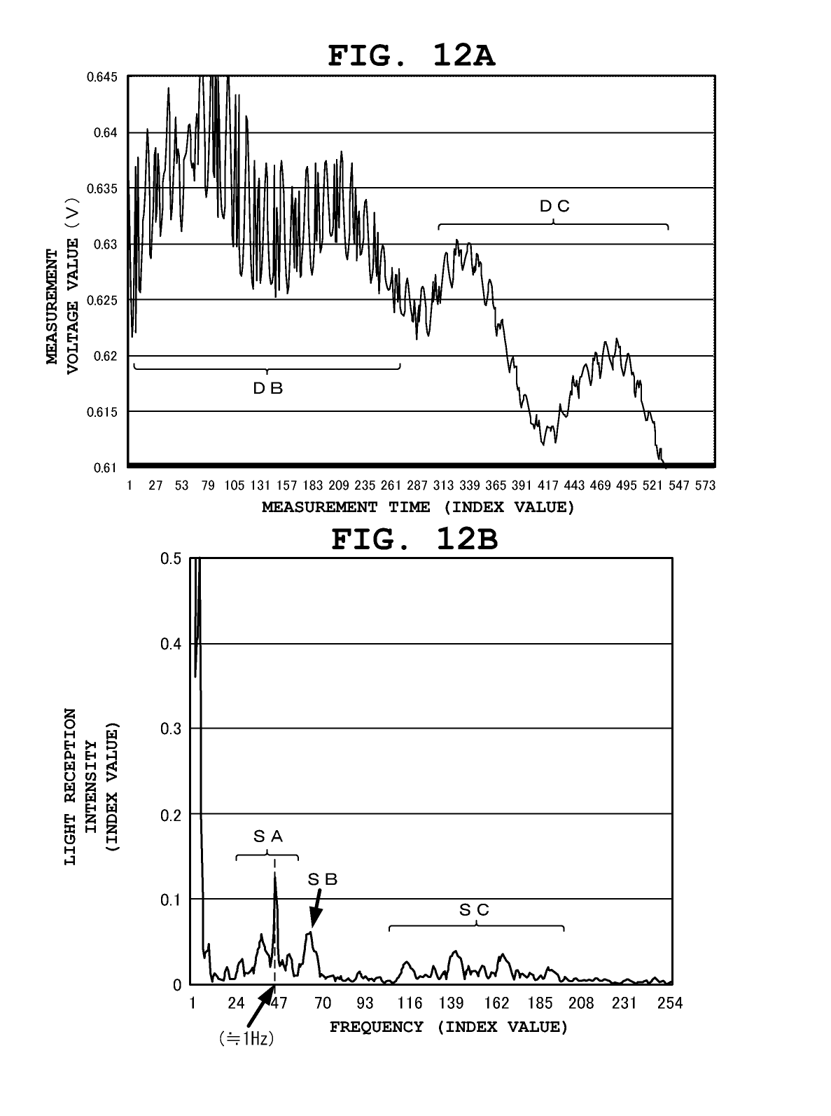

FIG. 9 is a flowchart of an example of the method of judging an appropriate combination of a light-receiving element(s) and light-emitting elements applied to the present specific example. FIG. 10A. FIG. 10B, FIG. 11A, FIG. 11B, FIG. 12A and FIG. 12B are diagrams each depicting an example of measurement data obtained by the pulse data detecting method and analysis data obtained by frequency analysis, according to the present specific example. Here, FIG. 10A and FIG. 10B depict measurement data (pulse wave data based on the output from the light-receiving element) with a sufficiently high S/N ratio of pulse components and in a favorable measurement state and analysis data obtained by frequency analysis thereof, respectively. FIG. 11A and FIG. 11B depict measurement data (pulse wave data based on the output from the light-receiving element) which prevents an S/N ratio of pulse components from being sufficiently ensured because of mixed noise due to, for example, ambient light and a motion of the human body causes a small signal amplitude, and analysis data obtained by frequency analysis thereof, respectively. FIG. 12A and FIG. 12B depict measurement data (pulse wave data based on the output from the light-receiving element) which affects to the extent that pulse components cannot be judged because of mixed significant noise due to, for example, a motion of the human body such as waving the hand or arm, and analysis data obtained by frequency analysis thereof, respectively. In FIG. 10A, FIG. 11A, and FIG. 12A, the horizontal axis represents index values each indicating a measurement time (a value obtained by converting elapsed time based on a specific index), and the vertical axis represents measurement voltage values. An output from the light-receiving element A is not limited to a voltage of an output signal (a measurement voltage value), but may be another measurement value such as a current. Also, in FIG. 10B, FIG. 11B and FIG. 12B, the horizontal axis represents index values each representing a frequency component (a value obtained by converting each frequency based on a specific index), and the vertical axis represents magnitudes of signal components in each frequency (a value obtained by converting light reception intensity at each frequency based on a specific index).

That is, at Step S400 according to the present first scheme, by following the flowchart depicted in FIG. 9, the CPU 11 first reads out the light-receiving element A and the light-emitting element B stored in the memory 12 at Step S412 and the Step S414. Here, the variable A specifying a light-receiving element and the variable B specifying a light-emitting element each have an initial value of 1. Next at Step S416, for the output value (sensor data) in the combination of the light-receiving element(s) A and the light-emitting elements B, the CPU 11 calculates distribution data of light reception intensity for each frequency component by Fourier transform. The CPU 11 stores the calculated distribution data of light reception intensity for each frequency component in a predetermined storage area of the memory 12.

Here, the calculated distribution data of light reception intensity for each frequency component is specifically described. Here, for convenience of description, actual measurement data with a sufficiently high S/N ratio of pulse components included in the obtained measurement data and in a favorable measurement state is used for description. The measurement data in the combination of the specific light-receiving element(s) A and the light-emitting elements B stored in the memory 12 is represented, for example, as in FIG. 10A. In FIG. 10A, regularly-repeated small waveforms PA each represent one pulse. In pulses of a person in a resting state, the pitch (time width) of one waveform is approximately equal to one second in general. Also, in the drawing, a large change (a dotted arrow in the drawing) PB of the measurement data formed of continuation of the small waveforms PA indicating pulses is due to a motion of the human body during measurement or the like. Also, the distribution data of light reception intensity for each frequency component obtained by Fourier transform of the measurement data depicted in FIG. 10A is represented, for example, as in FIG. 10B.

Next at Step S418, in the distribution data of light reception intensity for each frequency component, the CPU 11 extracts frequency components indicating peak values (maximum values) and its integer q-fold components (q=2, 3, 4, . . . ) as pulse components. That is, as depicted in FIG. 10B, in the distribution data obtained by Fourier transform, the result is obtained such that, for example, a peak XA with an extremely-high, maximum light reception intensity (index value) appears at a frequency position of approximately 1 Hz (an index value of approximately 42 on the horizontal axis) and peaks XB, XC, XD, . . . each with a light reception intensity sufficiently lower than that of the peak XA appear at positions that are approximately integer multiples of the frequency of the peak XA. Here, the peak XA is a component corresponding to a pulse, and the peaks XB, XC, XD, . . . are components (non-abnormal values) corresponding to second, third-order, fourth-order, . . . , harmonics of the peak XA. Therefore, when noise components are hardly mixed in the obtained measurement data, the S/N ratio of the pulse components is sufficiently high, and the measurement state is favorable, the component corresponding to the peak XA due to pulses or components corresponding to the peaks XA, XB, XC, XD, . . . are extracted and removed from the distribution data as pulse components, whereby only the noise components included in the measurement data can be extracted.

Next at Step S420, the CPU 11 judges whether the intensity of the data obtained by excluding the pulse components extracted at Step S418 described above (that is, noise components) from the distribution data obtained by Fourier transform is equal or larger than a certain value (threshold) set in advance. At Step S420, when the intensity of the noise components is equal to or larger than the certain value (YES at Step S420), the CPU 11 judges and excludes the combination of the light-receiving element(s) A and the light-emitting elements B as inappropriate (not being an appropriate combination) at Step S422, and performs processing at Step S428 onward, which will be described further below.

For example, when the signal amplitude of the measurement data is small and a sufficient S/N ratio cannot be ensured as depicted in FIG. 11A and FIG. 11B or when noise mixture is significant and pulse components cannot be distinguished as depicted in FIG. 12A and FIG. 12B, the CPU 11 judges the combination at this moment as inappropriate.

Specifically, in the measurement data depicted in FIG. 11A, noises are slightly included in pulse waveforms DA as a whole. Also, the signal amplitude of each waveform is very small compared with the measurement data depicted in FIG. 10A described above. Furthermore, entire change tendencies of the measurement data are also influenced by low-frequency noises. On the other hand, in the measurement data depicted in FIG. 12A, measurement data DB on a front half (a left half of the drawing) has very large noise mixed therein, and pulse waveforms can hardly be distinguished. Still further, in measurement data DC on a latter half (a right half of the drawing), mixture of large noise is solved. However, noises are slightly included in pulse waveforms, and the signal amplitude of each waveform is very small compared with the measurement data depicted in FIG. 10A described above.

In the distribution data of light reception intensity for each frequency component obtained by Fourier transform of the measurement data, as depicted in FIG. 11B and FIG. 12B, peak components SA to some extent near a frequency corresponding to the pulses can be detected. However, compared with the analysis data depicted in FIG. 10 described above, there are many unstable factors (such as mixture of a plurality of peaks and the presence of a nearby noise component SB). Therefore, it is also difficult to specify a frequency corresponding to the pulse from the peak components SA. Moreover, it is difficult to distinguish harmonic components of pulse components due to mixture of noise components SC.

Therefore, when the signal amplitude of the measurement data is small and a sufficient S/N ratio cannot be ensured or when noise mixture is significant and pulse components cannot be distinguished, pulse components cannot be removed from the distribution data. Or, even if pulse components can be removed from the distribution data, the intensity of the noise components is relatively strong and is equal to or larger than a certain value (threshold). Accordingly, the CPU 11 judges the combination of the light-receiving element(s) A and the light-emitting elements B set at this moment as inappropriate. Here, by taking one third of the light reception intensity in the frequency component indicating the peak value (maximum value) as a threshold, when the intensity of the data obtained by excluding the pulse components from the distribution data exceeds this threshold, the CPU 11 judges that noise is mixed in each frequency component to the extent that pulse components cannot be distinguished.

On the other hand, when the intensity of the noise components is smaller than the certain value (threshold) (NO at Step S420), the CPU 11 judges at Step S424 whether the light reception intensity in the frequency component indicating the peak value (maximum value) is maximum in the combinations of the light-receiving element(s) A and the light-emitting elements B so far. That is, the CPU 11 judges whether the light reception intensity in the frequency component of the peak XA corresponding to the pulse is maximum among the light reception intensities of peaks corresponding to the pulses extracted from the combinations of the light-receiving element(s) A and the light-emitting elements B set in the measurements so far.

Then at Step S424, when the light reception intensity in the frequency component indicating the peak value is maximum among the light reception intensities in the combinations so far (YES at Step S424), the CPU 11 judges that the combination of the light-receiving element(s) A and the light-emitting elements B at this moment is appropriate (an appropriate combination) at Step S426. The CPU 11 then sets this combination as one of appropriate combination candidates, and performs processing at Step S428 onward, which will be described further below. That is, when the light reception intensity in the frequency component of the peak XA is maximum of all measurements so far, the CPU 11 sets the combination of the light-receiving element(s) A and the light-emitting elements B at this moment as one of appropriate combination candidates, associates this combination with the light reception intensity at the peak XA, and temporarily stores the resultant data in a predetermined storage area of the memory 12. As such, the processing at Step S420 and S424 substantially corresponds to processing of judging whether pulse data is appropriate based on the S/N ratio.

On the other hand, at Step S424, when the light reception intensity in the frequency component of the peak value is not maximum (NO at Step S424), the CPU 11 increments the variable B specifying a light-emitting element by 1 (B+1 to B=2) at Step S428. Then at Step S430, when the incremented variable B is not larger than M indicating the maximum number of light-emitting elements, the CPU 11 returns to Step S414. As a result, for an output value (sensor data) in a combination of a newly specified light-emitting element B (=2) and the light-receiving element A (=1), a series of processing to which the above-described frequency analysis scheme by Fourier transform is applied (the method of judging an appropriate combination of light-emitting elements B and a light-receiving element(s) A) is repeated. That is, for an output value (sensor data) from the light-receiving element A (=1) when the light-emitting element B is changed to 1, 2, . . . , M, the CPU 11 performs frequency analysis by Fourier transform and judges an appropriate combination of light-emitting elements B and a light-receiving element(s) A.

Then at Step S430, when the variable B is larger than M indicating the maximum number of light-emitting elements, the CPU 11 increments the variable A specifying a light-receiving element by 1 (A+1 to A=2) at Step S432. Then, at Step S434, when the incremented variable A is not larger than N indicating the maximum number of light-receiving elements, the CPU 11 returns to Step S412. As a result, for an output value (sensor data) in a combination of the light-emitting element B (=1) and a newly specified light-receiving element A (=2), a series of processing to which the above-described frequency analysis scheme by Fourier transform is applied (the method of judging an appropriate combination of light-emitting elements B and a light-receiving element(s) A) is repeated. That is, for an output value (sensor data) from the light-receiving element A (=2) when the light-emitting element B is changed to 1, 2, . . . , M, the CPU 11 performs frequency analysis by Fourier transform and judges an appropriate combination of light-emitting elements B and a light-receiving element(s) A. By repeatedly performing this series of processing for each combination of the light-emitting element B (=1, 2, 3, . . . , M) and the light-receiving element A (=1, 2, 3, . . . , N), the latest, most appropriate combination candidate is stored in the memory 12 for update.

Then at Step S434, when the variable A is larger than N indicating the maximum number of light-receiving elements, the CPU 11 registers the latest (current) appropriate combination candidate stored in the memory 12 as an appropriate combination at Step S436, and stores the combination in a predetermined storage area of the memory 12. Thereafter, in the flowchart depicted in FIG. 8, processing at Step S340 onward is performed.

That is, by the processing of judging an appropriate combination of a light-receiving element(s) A and light-emitting elements B at Step S400 to which the above-described first scheme is applied, among combinations of a light-receiving element(s) A and light-emitting elements B from which measurement data and analysis data with a high S/N ratio and in a favorable measurement state can be obtained, a combination with the highest S/N ratio is judged and registered as the most appropriate combination as depicted in FIG. 10A and FIG. 10B, for example. On the other hand, for example, as depicted in FIG. 11A, FIG. 11B, FIG. 12A, and FIG. 12B, measurement data with a low S/N ratio and in a measurement state with a significant noise influence is excluded.

Next at Step S340, based on the appropriate combination judged at Step S400 described above, the CPU 11 determines the light-receiving element A and the light-emitting element B to be used for pulse measurement. Next, at Step S342, in the combination of the light-receiving element(s) A and the light-emitting elements B, the CPU 11 performs computation processing on the output value (sensor data) from the light-receiving element A. Furthermore, at Step S344, the pulse rate calculating section 18 calculates a pulse rate. Here, at Step S345, the CPU 11 judges whether an error is present in the pulse rate calculation processing (or whether the calculated pulse rate is adequate). When an error is present in the pulse rate calculation processing (YES at Step S345), the CPU 11 judges that the currently-set combination of the light-receiving element(s) A and the light-emitting elements B is inappropriate, and returns to Step S310, repeating the above-described series of processing of judging an appropriate combination described above (Step S310 to Step S340). On the other hand, when an error is not present in the pulse rate calculation processing (NO at Step S345), the CPU 11 outputs the calculated pulse rate to the display section 19 at Step S346. Next, at Step S348, the display section 19 displays the calculated pulse rate as pulse data. The calculated pulse rate is also associated with the combination of the light-receiving element(s) A and the light-emitting elements B and time data at the time of measurement, etc., and stored in a predetermined storage area of the memory 12.

Next at Step S350, the CPU 11 judges whether an end instruction is provided to the operating section 10 from the user. When an end instruction is not provided (NO at Step S350), the CPU 11 returns to Step S342, repeating the above-described processing of calculating a pulse rate. On the other hand, when an end instruction is provided from the user (YES at Step S350), the CPU 11 performs predetermined end processing (such as storing the pulse rate and discarding measurement data) at Step S352, and then ends the processing.

As such, in the present specific example, among one or a plurality of light-receiving elements and the plurality of light-emitting elements, the combination of a light-receiving element(s) and light-emitting elements to be used for pulse measurement is sequentially changed, whereby an appropriate combination from which an output with a favorable S/N ratio is determined based on the output from the light-receiving element(s) in each combination. As a result, according to the present specific example, an appropriate output level can be obtained regardless of the state of placement of the pulse data detecting apparatus 1 on the human body, and whereby stable and reliable pulse measurement can be performed.

Also in the present specific example, the combination of a light-receiving element(s) and light-emitting elements registered (stored) in advance, that is, for example, the appropriate combination of a light-receiving element(s) and light-emitting elements determined in a previous measurement and registered is set as a default state or an initial state in the next pulse measurement onward. As a result, according to the present specific example, pulse measurement can be performed by using the combination of the light-receiving element(s) and the light-emitting elements registered in advance until the obtained measurement data is judged as inappropriate. Therefore, processing for determining an appropriate combination can be omitted and whereby a user-friendly measuring apparatus with reduced process load and expeditious measurement processing can be provided.

In the present specific example, the case is described where a frequency analysis scheme by Fourier transform is applied as a method of judging an appropriate combination of a light-receiving element(s) and light-emitting elements. However, the present invention is not limited thereto. That is, in the present invention, another scheme other than Fourier transform may be applied as long as frequency analysis is applied to judge the quality of an output signal (for example, an S/N ratio) from the light-receiving element.

Furthermore, in the present specific example, the case is described where the method of judging an appropriate combination of a light-receiving element(s) and light-emitting elements is applied to the series of processing depicted in the flowchart of FIG. 3 of the pulse data detecting method in the above-described first embodiment. However, the present invention is not limited thereto. That is, the method of judging an appropriate combination applied to the present invention may be applied to the series of processing depicted in the flowchart of FIG. 4 or FIG. 5 in the above-described first embodiment or the flowchart of FIG. 6 in the second embodiment.

(Second Scheme)

Next, another example of scheme applicable to Step S200 in the above-described specific example is described.

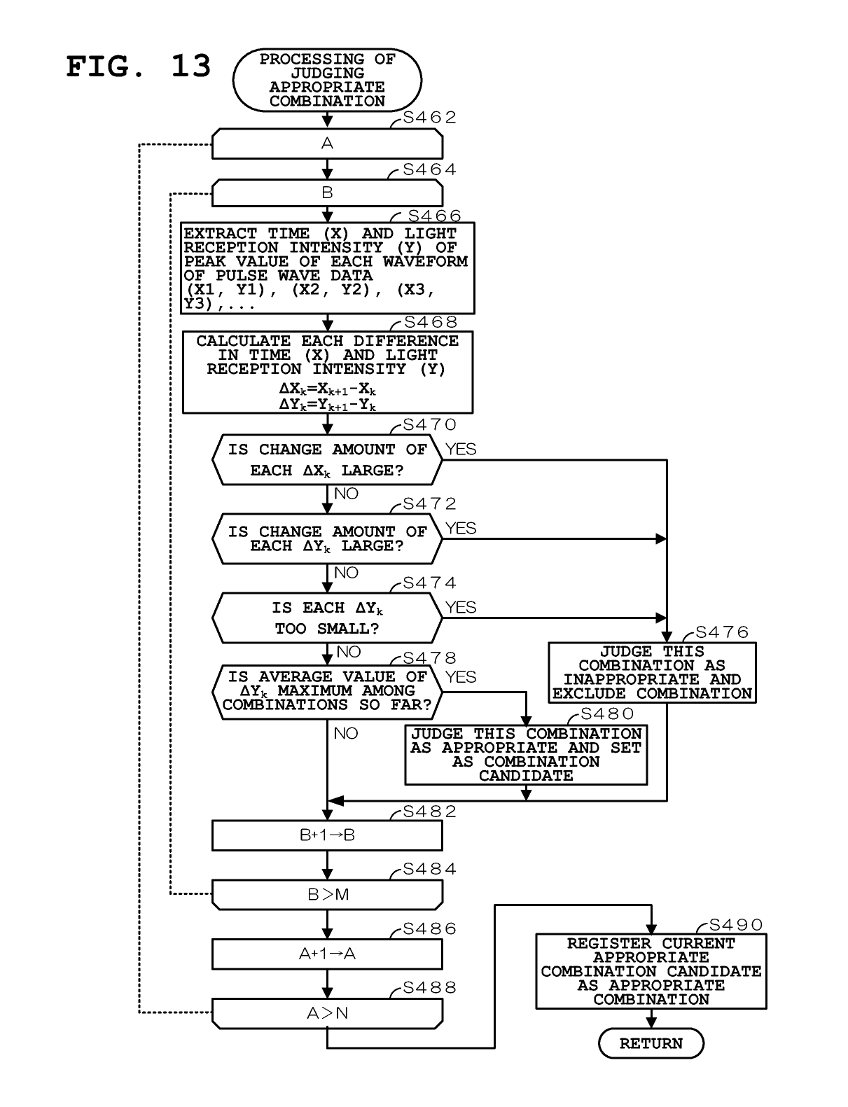

FIG. 13 is a flowchart of another example of the method of judging an appropriate combination of a light-receiving element(s) and light-emitting elements applied to the present specific example. Here, description is made by referring to the processing procedure of the above-described specific example (the flowchart depicted in FIG. 8) and the measurement data obtained in the processing procedure (pulse wave data based on the output from the light-receiving element depicted in FIG. 10A, FIG. 11A, and FIG. 12A).

In the method of judging an appropriate combination of a light-receiving element(s) and light-emitting elements in the above-described first scheme, the case is described where the measurement data is subjected to Fourier transform and, based on its analysis data, processing of judging an appropriate combination is performed. In the present second scheme, processing of judging an appropriate combination is performed based on a time of the output value (sensor data) in the measurement data and a change amount of light reception intensity.

That is, at Step S400 according to the second scheme applied to the above-described specific example (the flowchart depicted in FIG. 8), the CPU 11 performs processing according to the flowchart depicted in FIG. 13. First at Step S462 and Step S464, the CPU 11 reads out the light-receiving element A and the light-emitting element B stored in the memory 12. Next at Step S416, the CPU 11 extracts from measurement data (pulse wave data) for a predetermined time a time (X) and a light reception intensity (Y) of a peak value of each waveform (refer to the waveforms PA in FIG. 10A) that increases and decreases. Here, the peak value of each waveform is found by, for example, differentiating the light emission intensity (Y) with respect to the time (X). The CPU 11 associates the time (X) and the light reception intensity (Y) of the peak value of each waveform, and temporarily stores the result in the memory 12 in the form of (X1, Y1), (X2, Y2), (X3, Y3), . . . .

Next at Step S468, the CPU 11 calculates a difference ΔXₖ=Xₖ₊₁−Xₖ (k=1, 2, 3, . . . ) between the times (X) of the peak values of adjacent waveforms and a difference ΔYₖ=Yₖ₊₁−Yₖ (k×1, 2, 3, . . . ) between the light reception intensities (Y) of these waveforms, and temporarily stores the result in the memory 12 as difference data. Here, the difference ΔXₖ in time (X) of the peak values corresponds to a pitch between adjacent waveforms, and the difference ΔYₖ in light reception intensity (Y) corresponds to the amplitude of each waveform. The difference ΔXₖ in time (X) of the peak values is not limited to the one using peak values between waveforms as long as the different is to derive a time corresponding to a pitch between waveforms.

Next at Step S470, the CPU 11 judges whether a change amount (or dispersion) of each difference ΔXₖ in time (X) of the peak values calculated for adjacent waveforms at Step S468 is larger than a certain value set in advance (threshold). When the change amount of each difference ΔXₖ is larger than the certain value (YES at Step S470), the CPU 11 judges at Step S476 that the combination of the light-receiving element(s) A and the light-emitting elements B at this moment is inappropriate (is not an appropriate combination) and excludes the combination, and then performs processing at Step S482 onward, which will be described further below.

For example, when very large noise are mixed and pulse waveforms can be hardly distinguished as depicted in the measurement data DB of FIG. 12A, each difference ΔXₖ in time (X) of the peak values of adjacent waveforms may be large. Also, when noises are slightly included in pulse waveforms as depicted in the waveforms DA of FIG. 11A and the measurement data DC of FIG. 12A, each difference ΔXₖ in time (X) of the peak values of waveforms may be small irregularly. Thus, in order to exclude the measurement data in a measurement state as described above, the CPU 11 judges the combination of the light-receiving element(s) A and the light-emitting elements B set at this moment as inappropriate.

On the other hand, at Step S470, when the change amount of each difference ΔXₖ in time (X) of the peak values of waveforms is not larger than the certain value (NO at Step S470), the CPU 11 judges at Step S472 whether the change amount (or dispersion) of each difference ΔYₖ in light reception intensity (Y) of adjacent waveforms is larger than a certain value set in advance (threshold). When the change amount of each difference ΔYₖ is larger than the certain value (YES at Step S472), the CPU 11 judges at Step S476 that the combination of the light-receiving element(s) A and the light-emitting elements B at this moment is inappropriate and excludes the combination, and then performs processing at Step S482 onward, which will be described further below.

For example, when very large noise is mixed and the amplitude of each waveform is greatly changed as depicted in the measurement data DB of FIG. 12A, the change amount of each difference ΔYₖ in light reception intensity (Y) of adjacent waveforms is large. Therefore, in order to exclude the measurement data in a measurement state as described above, the CPU 11 judges the combination of the light-receiving element(s) A and the light-emitting elements B set at this moment as inappropriate.

On the other hand, at Step S472, when the change amount of each difference ΔYk in light reception intensity (Y) of waveforms is not larger than the certain value (NO at Step S472), the CPU judges at Step S474 whether each difference ΔYₖ in light reception intensity (Y) of waveforms is extremely smaller than a certain value (threshold) set in advance (that is, too small). When each difference ΔYk in light reception intensity (Y) is too small (YES at Step S474), the CPU 11 judges at Step S476 the combination of the light-receiving element(s) A and the light-emitting elements B at this moment as inappropriate and excludes the combination, and then performs processing at Step S482 onward, which will be described further below.

For example, when the output signal from the light-receiving element A is weak (the measurement voltage is low) and the amplitude of each waveform is very small as depicted in the measurement data DA of FIG. 11A, the difference ΔYk in light reception intensity (Y) of adjacent waveforms is extremely small. Therefore, in order to exclude the measurement data in a measurement state as described above, the CPU 11 judges the combination of the light-receiving element(s) A and the light-emitting elements B set at this moment as inappropriate.

On the other hand, at Step S474, when the difference ΔYₖ in light reception intensity (Y) is not too small (NO at Step S474), the CPU 11 judges at Step S478 whether an average value of the differences ΔYₖ in light reception intensity (Y) in the measurement data is maximum among average values of differences ΔYₖ in respective combinations of the light-receiving element(s) A and the light-emitting elements B set in the measurements so far.

Then at Step S478, when the average value of the differences ΔYₖ in light reception intensity (Y) is maximum among those of the combinations so far (YES at Step 478), the CPU 11 judges at Step S480 that the combination of the light-receiving element(s) A and the light-emitting elements B at this moment is appropriate (an appropriate combination) and sets this combination as one of appropriate combination candidates, and then performs processing at Step S482 onward, which will be described further below. That is, when the average value of the differences ΔYₖ in light reception intensity (Y) is maximum among the measurements so far, the CPU 11 sets the right-receiving element(s) A and the light-emitting elements B at this moment as one of appropriate combination candidates, associates the candidate with the average value of the differences ΔYₖ in light reception intensity (Y), and temporarily stores the result in a predetermined storage area of the memory 12.

On the other hand, at Step S478, when the average value of the differences ΔYₖ in light reception intensity (Y) is not maximum (NO at Step S478), the CPU 11 increments the variable B specifying a light-emitting element by 1 (B+1 to B−2) at Step S482. Then, at Step S484, when the incremented variable B is not larger than M indicating the maximum number of light-emitting elements, the CPU 11 returns to Step S464. As a result, for output values (sensor data) in a combination of a newly specified light-emitting element B (=2) and the light-receiving element A (=1), the above-described series of processing (the method of judging an appropriate combination of light-emitting elements B and a light-receiving element(s) A) is repeated, to which the analysis scheme based on the difference ΔXₖ in time (X) of the peak values of adjacent waveforms and the difference ΔYₖ in light reception intensity (Y) of the waveforms is applied. That is, for output values (sensor data) from the light-receiving element A (=1) when the light-emitting element B is changed to 1, 2, . . . , M, the CPU 11 performs an analysis based on the difference ΔXₖ in time (X) of the peak values of waveforms and the difference ΔYₖ in light reception intensity (Y) thereof to judge an appropriate combination of light-emitting elements B and a light-receiving element(s) A.

Then at Step S484, when the variable B is larger than M indicating the maximum number of light-emitting elements, the CPU 11 increments the variable A specifying a light-receiving element by 1 (A+1 to A=2) at Step S486. Then, at Step S488, when the incremented variable A is not larger than N indicating the maximum number of light-receiving elements, the CPU 11 returns to Step S462. As a result, for output values (sensor data) in a combination of the light-emitting element B (=1) and a newly specified light-receiving element A (=2), the above-described series of processing (the method of judging an appropriate combination of light-emitting elements B and a light-receiving element(s) A) is repeated, to which the analysis scheme based on the difference ΔXₖ in time (X) of the peak values of adjacent waveforms and the difference ΔYₖ in light reception intensity (Y) of the waveforms is applied. That is, for output values (sensor data) from the light-receiving element A (=2) when the light-emitting element B is changed to 1, 2, . . . , M, the CPU 11 performs an analysis based on the difference ΔXₖ in time (X) of the peak values of waveforms and the difference ΔYₖ in light reception intensity (Y) thereof to judge an appropriate combination of light-emitting elements B and a light-receiving element(s) A. By repeatedly performing this series of processing for each combination of the light-emitting element B (=1, 2, 3, . . . M) and the light-receiving element A (=1, 2, 3, . . . , N), the latest and most appropriate combination candidate is stored in the memory 12 for update.

Then at Step S488, when the variable A is larger than N indicating the maximum number of light-receiving elements, the CPU 11 registers, at Step S490, the latest (current) appropriate combination candidate stored in the memory 12 as an appropriate combination, and stores the combination in a predetermined storage area of the memory 12.

At Step S488, when the variable A is larger than the maximum value N, as with the above-described first scheme, the CPU 11 registers, at Step S490, the latest (current) appropriate combination candidate stored in the memory 12 as the most appropriate combination, and stores the combination in a predetermined storage area of the memory 12. Thereafter, the processing at Step S340 onward is performed in the flowchart of FIG. 8.

That is, by the processing of judging an appropriate combination of a light-receiving element(s) A and the light-emitting element B at Step S400 to which the above-described second scheme is applied, a combination with the largest amplitude average value is judged and registered as the most appropriate combination, among the combinations of light-receiving elements A and light-emitting elements B from which measurement data can be obtained where the pulse waveform pitch and amplitude are uniform and the amplitude is sufficiently large as depicted in, for example, FIG. 10A. On the other hand, measurement data where the waveform pitch and amplitude are not uniform due to noise mixture and measurement data with a very small amplitude as depicted in, for example, FIG. 11A and FIG. 12A, are excluded. In the judgment processing using the difference ΔXₖ in time (X) of the peak values of waveforms and the difference ΔYₖ in light reception intensity (Y) thereof at Step S470, S472, and S474 described above, the CPU 11 applies as thresholds, for example, a pulse waveform pitch and amplitude obtained by measuring a pulse for a predetermined period.

As has been described above, according to the present specific example, among one or a plurality of light-receiving elements and a plurality of light-emitting elements, the combination of a light-receiving element(s) and light-emitting elements to be used for pulse measurement is sequentially changed, whereby an appropriate combination from which an output with a favorable pulse wave pitch and amplitude can be obtained is determined based on an output from the light-receiving element in each combination. As a result, according to the present specific example, an appropriate output level can be obtained regardless of the state of placement of the pulse data detecting apparatus 1 on the human body, and thereby stable and reliable pulse measurement can be performed.

Also in the present specific example, by computation processing of performing calculation of the difference ΔXₖ in time (X) of peak values of adjacent waveforms included in measurement data and the difference ΔYₖ in light reception intensity (Y) thereof and making comparison between each calculated difference and a certain value (threshold), an appropriate combination of a light-receiving element(s) and light-emitting elements is judged. As a result, according to the present specific example, the processing of determining an appropriate combination of a light-receiving element(s) and light-emitting elements can be performed by simple computation processing, whereby a user-friendly measuring apparatus with reduced process load and expeditious measurement processing can be provided. Here, in the present second scheme, an appropriate combination of a light-receiving element(s) and light-emitting elements can be judged basically as long as measurement data including at least waveforms of two pulses is present. In actual pulse measurement, measurement data including several to several tens of waveforms is preferable. In this case, an operation of measuring and capturing an output from the light-receiving element is performed at a time of, for example, several to several tens of seconds.

While the present invention has been described with reference to the preferred embodiments, it is intended that the invention be not limited by any of the details of the description therein but includes all the embodiments which fall within the scope of the appended claims.

1. A pulse data detecting apparatus comprising:
a plurality of light-emitting elements which irradiate a body to be measured with light;
a light emission control section which performs control of causing the plurality of light-emitting elements to emit light in a plurality of light emission patterns;
a light-receiving element which receives reflected light when the body to be measured is irradiated by the plurality of light-emitting elements in the plurality of light emission patterns and outputs a signal for each of the light emission patterns;
a combination determining section which determines, as an appropriate combination, a combination of any of the plurality of light emission patterns and the light-receiving element satisfying an adequate condition, based on the signal outputted from the light-receiving element; and
a pulse data output section which outputs pulse data based on the signal outputted from the light-receiving element, by the combination of the light emission patterns and the light-receiving element determined by the combination determining section as the appropriate combination.

2. The pulse data detecting apparatus according to claim 1, wherein the light emission control section causes the plurality of light-emitting elements to emit light in a plurality of light emission patterns by controlling, among the plurality of light-emitting elements, a number of light-emitting elements to be lit up, a position of each of the light-emitting elements to be lit up, or a light emission amount of each of the light-emitting elements to be lit up independently or in combination.

3. The pulse data detecting apparatus according to claim 1, wherein the light emission control section performs control of causing the plurality of light-emitting elements to emit light in the plurality of light emission patterns by sequentially and simultaneously lighting up at least two or more of the plurality of light-emitting elements in different combinations, and
wherein the combination determining section, every time the at least two or more of the light-emitting elements are lit up simultaneously in different combinations, determines, as the appropriate combination, a combination of any of the plurality of light emission patterns by simultaneous lighting-up the at least two or more of the light-emitting elements and the light-receiving element satisfying the adequate condition, based on the signal outputted from the light-receiving element.

4. The pulse data detecting apparatus according to claim 1, wherein the light emission control section performs control of causing the plurality of light-emitting elements to emit light in the plurality of light emission patterns by sequentially lighting up any one of the plurality of light-emitting elements,
wherein the combination determining section, every time the any one of the plurality of light-emitting elements is sequentially lit up, determines, as the appropriate combination, a combination of any of the plurality of light emission patterns by the any one of the plurality of light-emitting elements and the light-receiving element satisfying the adequate condition, based on the signal outputted from the light-receiving element,
wherein the light emission control section further causes the plurality of light-emitting elements to emit light in the plurality of light emission patterns by causing at least two or more of the plurality of light-emitting elements to sequentially light up simultaneously in different combinations when the combination determining section cannot determine the appropriate combination of the light emission pattern by the any one of the plurality of light-emitting elements and the light receiving element, and
wherein the combination determining section, every time the at least two or more of the plurality of light-emitting elements are lit up simultaneously in different combinations, determines, as the appropriate combination, a combination of any of the plurality of light emission patterns by the combination of the at least two or more of the plurality of light-emitting elements and the light-receiving element satisfying the adequate condition, based on the signal outputted from the light-receiving element.

5. The pulse data detecting apparatus according to claim 1, wherein the light emission control section performs control of causing the plurality of light-emitting elements to emit light in the plurality of light emission patterns by sequentially and simultaneously lighting up at least two or more of the plurality of light-emitting elements in different combinations and with different light amounts, and
wherein the combination determining section, every time the at least the two or more of the light-emitting elements are lit up simultaneously in different combinations and with different light amounts, determines, as the appropriate combination, a combination of any of the plurality of light emission patterns by simultaneous lighting up with any of the light amounts of the at least two or more of the plurality of light-emitting elements and the light-receiving element satisfying the adequate condition, based on the signal outputted from the light-receiving element.

6. The pulse data detecting apparatus according to claim 1, wherein a plurality of the light-receiving elements are provided around the plurality of light-emitting elements,
wherein the combination determining section determines, as the appropriate combination, a combination of any of the plurality of light emission patterns and any of the plurality of light-receiving elements satisfying the adequate condition, based on the signal outputted from each of the plurality of the light-receiving elements, and
wherein the pulse data output section outputs the pulse data based on the signal outputted from the determined light-receiving element, by the light emission pattern determined by the combination determining section.

7. The pulse data detecting apparatus according to claim 1, wherein the light emission control section performs control of causing lighting up by sequentially decreasing light emission amounts of the determined light-emitting elements in the combination of the light emission pattern and the light-receiving element determined by the combination determining section, and
wherein the pulse data detecting apparatus further comprises a light emission amount determining section which, every time the light-emitting elements are caused to light up with the light emission amounts of the light-emitting elements sequentially decreased, determines, as appropriate light emission amount, lowest light emission amount of the light-emitting elements such that measurement of the pulse data can be performed with the signal outputted from the light-receiving element in the appropriate combination of the light emission pattern and the light-receiving element based on the signal outputted from the light-receiving element.

8. The pulse data detecting apparatus according to claim 1, wherein the combination determining section determines, as the appropriate combination, a combination of any of the plurality of light emission patterns and the light-receiving element satisfying the adequate condition, based on ratios between pulse signal components in a distribution of detection intensity for each frequency component of the signal outputted from the light-receiving element and noise components for each of combinations of the plurality of light emission patterns and the light-receiving element.

9. The pulse data detecting apparatus according to claim 8, wherein the combination determining section determines at least, as the appropriate combination, a combination of the light emission pattern and the light-receiving element with maximum one of the ratios between the pulse signal components and the noise components for each of the combinations of the plurality of light emission patterns and the light-receiving element.

10. The pulse data detecting apparatus according to claim 1, wherein the combination determining section determines, as the appropriate combination, a combination of any of the plurality of light emission patterns and the light-receiving element satisfying the adequate condition, based on change amounts of a pitch and an amplitude of each waveform of the signal outputted from the light-receiving element for each of the combinations of the plurality of light emission patterns and the light-receiving element.

11. The pulse data detecting apparatus according to claim 10, wherein the combination determining section determines at least, as the appropriate combination, a combination of the light emission pattern and the light-receiving element with one of the change amounts of the pitch and the amplitude of each waveform of the signal where an average value of the amplitudes is maximum, for each of the combinations of the plurality of light emission patterns and the light-receiving element.

12. The pulse data detecting apparatus according to claim 1, further comprising a combination storage section which stores a combination of any of the plurality of light emission patterns and the light-receiving element,
wherein the pulse data output section outputs the pulse data based on the signal outputted from the light-receiving element in the combination of the light emission patterns and the light-receiving element stored in advance in the combination storage section.

13. A pulse data detecting apparatus comprising:
a plurality of light-emitting elements which irradiate a body to be measured with light;
a light emission control section which performs control of light emission amounts of the plurality of light-emitting elements;
a light-receiving element which receives reflected light when the body to be measured is irradiated by the plurality of light-emitting elements with the light emission amounts controlled by the light emission control section and outputs a signal; and
a pulse data output section which outputs pulse data based on the signal outputted from the light-receiving element.

14. The pulse data detecting apparatus according to claim 13, further comprising a light emission amount storage section which stores the light emission amounts of the plurality of light-emitting elements,
wherein the pulse data output section outputs the pulse data based on the signal outputted from the light-receiving element with the light emission amounts of the plurality of light-emitting elements stored in advance in the light emission amount storage section.

15. A pulse data detecting method comprising:
a step of performing control of causing a plurality of light-emitting elements to emit light in a plurality of light emission patterns when irradiating a body to be measured with light by the plurality of light-emitting elements;
a step of receiving reflected light by a light-receiving element when the body to be measured is irradiated by the plurality of light-emitting elements in the plurality of light emission patterns, and converting the reflected light to a signal for each of the light emission patterns and outputting the signal;
a step of determining, as an appropriate combination, a combination of any of the plurality of light emission patterns and the light-receiving element satisfying an adequate condition, based on the signal outputted from the light-receiving element; and
a step of outputting pulse data based on the signal outputted from the light-receiving element, by the combination of the light emission patterns and the light-receiving element determined as the appropriate combination.

16. A pulse data detecting method comprising:
a step of performing control of light emission amounts of a plurality of light-emitting elements when irradiating a body to be measured with light by the plurality of light-emitting elements;
a step of receiving reflected light by a light-receiving element when the body to be measured is irradiated by the plurality of light-emitting elements with the light emission amounts controlled and converting the reflected light to a signal and outputting the signal; and
a step of outputting pulse data based on the signal outputted from the light-receiving element.

17. A non-transitory computer-readable storage medium having stored thereon a pulse data detection program that is executable by a computer, the program being executable by the computer to perform functions comprising:
processing for performing control of causing a plurality of light-emitting elements to emit light in a plurality of light emission patterns when irradiating a body to be measured with light by the plurality of light-emitting elements;
processing for receiving reflected light by a light-receiving element when the body to be measured is irradiated by the plurality of light-emitting elements in the plurality of light emission patterns, and converting the reflected light to a signal for each of the light emission patterns and outputting the signal;
processing for determining, as an appropriate combination, a combination of any of the plurality of light emission patterns and the light-receiving element satisfying an adequate condition, based on the signal outputted from the light-receiving element; and
processing for outputting pulse data based on the signal outputted from the light-receiving element, by the combination of the light emission patterns and the light-receiving element determined as the appropriate combination.

18. A non-transitory computer-readable storage medium having stored thereon a pulse data detection program that is executable by a computer, the program being executable by the computer to perform functions comprising:
processing for performing control of light emission amounts of a plurality of light-emitting elements when irradiating a body to be measured with light by the plurality of light-emitting elements;
processing for receiving reflected light by a light-receiving element when the body to be measured is irradiated by the plurality of light-emitting elements with the light emission amounts controlled, and converting the reflected light to a signal and outputting the signal; and
processing for outputting pulse data based on the signal outputted from the light-receiving element.
